(19) United States
(12) Patent Application Publication
(10) Pub. No.: US 2021/0015865 A1
(43) Pub. Date: Jan. 21, 2021

(54) CHIMERIC ENGULFMENT RECEPTORS AND USES THEREOF FOR NEURODEGENERATIVE DISEASES

(71) Applicant: CERO THERAPEUTICS, INC., South San Francisco, CA (US)

(72) Inventor: Daniel Mark COREY, Menlo Park, CA (US)

(21) Appl. No.: 17/040,341

(22) PCT Filed: Mar. 27, 2019
(86) PCT No.: PCT/US2019/024433

§ 371 Date: Sep. 22, 2020

Related U.S. Application Data
(60) Provisional application No. 62/649,472, filed on Mar. 28, 2018.

(51) Int. Cl. A61K 35/17; A61K 38/18; C07K 16/18; C12N 15/867; A61P 25/28

(52) U.S. Cl. CPC A61K 35/17; A61K 38/1841; C07K 16/18; C12N 2501/15; A61P 25/28; C07K 2317/53; C12N 15/867

(57) ABSTRACT

The present disclosure relates to chimeric engulfment receptor molecules, host cells modified to include the phagocytic engulfment molecules, and methods of making and using such receptor molecules and modified cells for the treatment of neurodegenerative diseases.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 200265_405WO_SEQUENCE_LISTING.txt. The text file is 94.8 KB, was created on Mar. 13, 2019, and is being submitted electronically via EFS-Web.

### BACKGROUND

Neurodegenerative diseases affects millions of people worldwide, and their prevalence is increasing, in part due to a general increase in lifespan. Neurodegenerative diseases are characterized by the progressive loss of neurons in the brain and/or spinal cord. Neurodegenerative diseases encompass a broad range of clinical diseases, and the clinical features depend upon the particular central nervous system region involved. Neurodegenerative diseases include Alzheimer's disease, amyotrophic lateral sclerosis, Friedreich's ataxia, Huntington's disease, Lewy body disease, Parkinson's disease, spinal muscular dystrophy, and prion diseases. Many of these diseases are found to share common cellular and molecular mechanisms, including abnormal accumulation and aggregation of specific proteins, which are typically deposited in intracellular inclusions or extracellular aggregates in specific brain regions. The aggregates are usually composed of fibrils containing misfolded proteins. For example, extracellular amyloid plaques composed of aggregated amyloid-peptide in specific cortical areas of the brain are a pathological marker for Alzheimer's Disease. An abundance of misfolded proteins is toxic to neurons, leading to cell injury and death. Moreover, several studies have suggested that protein aggregates, such as those composed of Tau, polyglutamine-containing proteins, and amyloid-β, are capable of spreading to other cells and brain regions, further contributing to disease progression.

Current treatments for neurodegenerative diseases are limited and typically aim to treat the symptoms only. None of the currently available therapies slow or stop the continued loss of neurons or decrease aberrant protein aggregation. There is a clear need for alternative therapies directed against neurodegenerative diseases. Presently disclosed embodiments address these needs and provide other related advantages.

### BRIEF DESCRIPTION OF THE DRAWINGS

FIG. 1 shows a schematic of an exemplary chimeric engulfment receptor (CER64) comprising: an extracellular domain comprising an amyloid-β specific scFv (BIIB037 scFv) and an IgG4 hinge region extracellular spacer domain; a Tim4 transmembrane domain; and a MERTK homeostatic engulfment signaling domain.

FIG. 2 shows a schematic for an in vitro engulfment assay of CER64 modified cells co-incubated with labeled amyloid-β1-42 peptide (“AB42”; SEQ ID NO:1), which is the predominant component of amyloid plaques in brains of people with Alzheimer's disease.

FIGS. 3A-3C show microscopy images showing in vitro engulfment of AB42 peptide oligomers by CER64 modified Ba/F3 host cells at 1.5 hours (FIG. 3A), 3 hours (FIG. 3B) post-incubation. Arrows indicate examples of engulfment events. FIG. 3C shows that Ba/F3 host cells transduced with EGFR as a control do not exhibit engulfment activity after 1.5 hours co-incubation with AB42 peptide oligomers.

FIG. 4: CER64 transduced Ba/F3 cells encoding a chimeric engulfment receptor targeting β-amyloid were co-incubated with fluorescently-labeled AB oligomers and β-amyloid fibrils for 1.5 hr at 37° C., washed twice, and the percentage of fluorescent⁺ Ba/F3 cells was quantified. Experiments were done in triplicate and the average values were plotted. Phagocytosis was determined as the percentage of fluorescent-positive cells in the Ba/F3 population.

FIG. 5A: shows microscopy images of human B cells which were activated with cytokine and transduced with a lentiviral cassette encoding-CER64, a chimeric engulfment receptor targeting β-amyloid. Transduced cells were co-incubated with fluorescently-labeled AB oligomers and β-amyloid fibrils for 1.5 hr at 37° C., washed twice, and the percentage of fluorescent transduced B cells quantified.

FIG. 5B shows Control B cells transduced with EGFR do not exhibit engulfment activity after 1.5 hour co-incubation with AB42 peptide oligomers.

FIG. 6: Human B cells were activated with cytokine and transduced with a lentiviral cassette encoding-CER64, a chimeric engulfment receptor targeting β-amyloid. Transduced cells were co-incubated with fluorescently-labeled AB oligomers and β-amyloid fibrils for 1.5 hr at 37° C., washed twice, and the percentage of fluorescent transduced B cells quantified. Experiments were done in triplicate and the average values were plotted. Phagocytosis was determined as the percentage of fluorescent-positive cells in the human B cell population.

FIGS. 7A-7B show microscopy images of in vitro phagocytosis of AB42 peptide oligomers/fibrils. Engulfed, fluorescent stained AB42 peptide (red) inside CER64 modified Ba/F3 cells are shown in the left image of FIG. 7A, while acidic lysosomes within the CER64 modified Ba/F3 cells are dyed green with LysoTracker® Green and shown in the right image of FIG. 7A. FIG. 7B shows an overlay of the two images from FIG. 7A, showing that the engulfed AB42 peptide is localized to phagolysosomal compartments (arrows) to undergo breakdown.

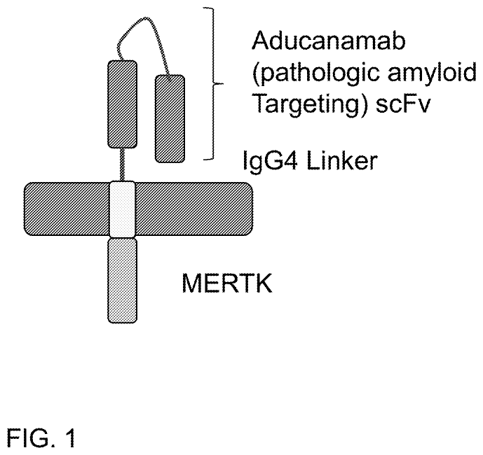

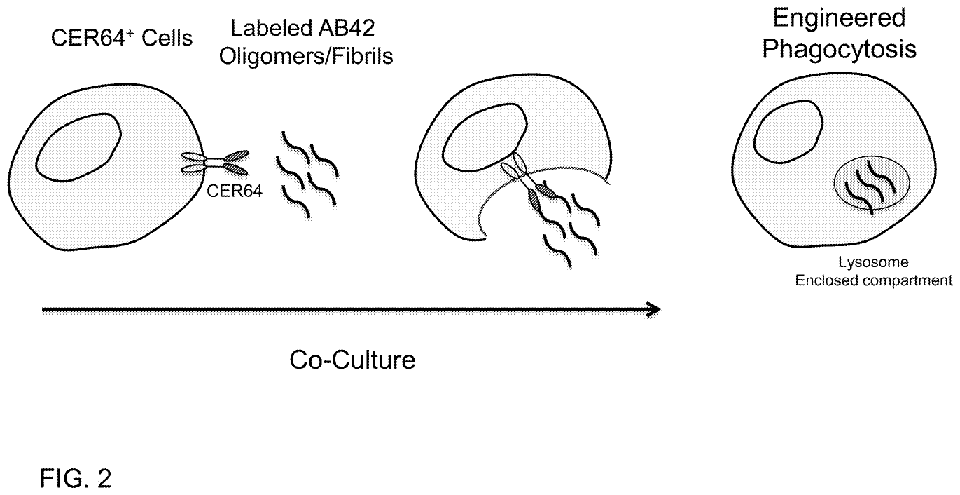

### DETAILED DESCRIPTION

The present disclosure generally relates to chimeric proteins comprising: (a) an extracellular domain comprising a binding domain that binds to a neurodegenerative disease antigen, (b) an engulfment signaling domain comprising a homeostatic engulfment signaling domain; and (c) a transmembrane domain positioned between and connecting the extracellular domain and the homeostatic engulfment signaling domain; and nucleic acid molecules encoding said chimeric proteins. In certain embodiments, the extracellular domain of the chimeric proteins described herein optionally includes an extracellular spacer domain positioned between and connecting the binding domain and transmembrane domain. Additionally, cells modified to express these chimeric proteins and methods and compositions for delivery of such modified cells to a subject in need thereof are provided.

The chimeric proteins of the present disclosure are referred to herein as a “chimeric engulfment receptor” or “chimeric engulfment receptors” (“CER” in the singular and “CERs” in the plural). Chimeric engulfment receptors described herein are capable of conferring an engulfment phenotype that is specific for a neurodegenerative disease antigen to a host cell that is modified to express said chimeric engulfment receptor. In certain embodiments, expression of a CER as described herein confers an engulfment phenotype to a host cell that does not naturally exhibit an engulfment phenotype. CERs of the present disclosure may be used to redirect engulfment specificity to target cells that express the targeted neurodegenerative disease antigen. CERs of the present disclosure may also be used to redirect engulfment specificity to target neurodegenerative disease antigens that are proteins (e.g., not bound on cell surface), extracellular protein aggregates, or antigenic particles. Thus, in certain embodiments, CER immunotherapy may be designed to target a neurodegenerative disease-associated antigen to clear diseased cells expressing the neurodegenerative disease antigen, or to reduce aberrant protein accumulation, in particular extracellular protein aggregates that are characteristic of neurodegenerative diseases.

Prior to setting forth this disclosure in more detail, it may be helpful to an understanding thereof to provide definitions of certain terms to be used herein.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

Terms understood by those in the art of antibody technology are each given the meaning acquired in the art, unless expressly defined differently herein. The term “antibody” is used in the broadest sense and includes polyclonal and monoclonal antibodies. An “antibody” may refer to an intact antibody comprising at least two heavy (H) chains and two light (L) chains inter-connected by disulfide bonds, as well as an antigen-binding portion (or antigen-binding domain) of an intact antibody that has or retains the capacity to bind a target molecule. An antibody may be naturally occurring, recombinantly produced, genetically engineered, or modified forms of immunoglobulins, for example intrabodies, peptibodies, nanobodies, single domain antibodies, SMIPs, multispecific antibodies (e.g., bispecific antibodies, diabodies, triabodies, tetrabodies, tandem di-scFV, tandem tri-scFv, ADAPTIR). A monoclonal antibody or antigen-binding portion thereof may be non-human, chimeric, humanized, or human, preferably humanized or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988). “Antigen-binding portion” or “antigen-binding domain” of an intact antibody is meant to encompass an “antibody fragment,” which indicates a portion of an intact antibody and refers to the antigenic determining variable regions or complementary determining regions of an intact antibody. Examples of antibody fragments include, but are not limited to, Fab, Fab′, F(ab′)2, and Fv fragments, Fab′-SH, F(ab′)₂, diabodies, linear antibodies, scFv antibodies, VH, and multispecific antibodies formed from antibody fragments. A “Fab” (fragment antigen binding) is a portion of an antibody that binds to antigens and includes the variable region and CH1 of the heavy chain linked to the light chain via an inter-chain disulfide bond. An antibody may be of any class or subclass, including IgG and subclasses thereof (IgG₁, IgG₂, IgG₃, IgG₄), IgM, IgE, IgA, and IgD.

The term “variable region” or “variable domain” refers to the domain of an antibody heavy or light chain that is involved in binding of the antibody to antigen.

The variable domains of the heavy chain and light chain (VH and VL, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The terms “complementarity determining region” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (HCDR1, HCDR2, HCDR3) and three CDRs in each light chain variable region (LCDR1, LCDR2, LCDR3).

As used herein, the terms “binding domain,” “binding region,” and “binding moiety” refer to a molecule, such as a peptide, oligopeptide, polypeptide, or protein that possesses the ability to specifically and non-covalently bind, associate, unite, recognize, or combine with a target molecule (e.g., Tau, β-amyloid, α-synuclein). A binding domain includes any naturally occurring, synthetic, semi-synthetic, or recombinantly produced binding partner for a biological molecule or other target of interest. In some embodiments, the binding domain is an antigen-binding domain, such as an antibody or functional binding domain or antigen-binding portion thereof. Exemplary binding domains include single chain antibody variable regions (e.g., domain antibodies, sFv, scFv, Fab), receptor ectodomains (e.g., TNF-α), ligands (e.g., cytokines, chemokines), or synthetic polypeptides selected for the specific ability to bind to a biological molecule.

A variety of assays are known for identifying binding domains of the present disclosure that specifically bind a particular target, as well as determining binding domain affinities, such as Western blot, ELISA, and BIACORE® analysis (see also, e.g., Scatchard et al., Ann. N.Y. Acad. Sci. 51:660, 1949; and U.S. Pat. Nos. 5,283,173, 5,468,614, or the equivalent). As used herein, “specifically binds” refers to an association or union of a binding domain, or a fusion protein thereof, to a target molecule with an affinity or Kₐ (i.e., an equilibrium association constant of a particular binding interaction with units of 1/M) equal to or greater than 10⁵ M⁻¹, while not significantly associating or uniting with any other molecules or components in a sample.

The terms “antigen” and “Ag” refer to a molecule that is capable of specifically binding to an antibody, receptor, ligand, polypeptide, or small molecule in a host organism. In certain embodiments, an antigen is capable of inducing an immune response. Macromolecules, including proteins, glycoproteins, peptides, and glycolipids, can serve as an antigen. An antigen may be from a host molecule (self-antigen or autoantigen) or a foreign molecule, including toxins, chemicals, bacteria, viruses, haptens, prions.

The term “epitope” or “antigenic epitope” includes any molecule, structure, amino acid sequence or protein determinant within an antigen that is specifically bound by a cognate immune binding molecule, such as an antibody or fragment thereof (e.g., scFv), T cell receptor (TCR), chimeric engulfment receptor, or other binding molecule, domain or protein. Epitopic determinants generally contain chemically active surface groupings of molecules, such as amino acids or sugar side chains, and can have specific three dimensional structural characteristics, as well as specific charge characteristics. An epitope may be a linear epitope or a conformational epitope.

As used herein, an “effector domain” is an intracellular portion of a fusion protein or chimeric receptor that can directly or indirectly promote a biological or physiological response in a cell expressing the effector domain when receiving the appropriate signal. In certain embodiments, an effector domain is part of a protein or protein complex that receives a signal when bound. In other embodiments, the effector domain is part of a protein or protein complex that binds directly to a target molecule, which triggers a signal from the effector domain. For example, in response to binding of the CER to a target molecule, the effector domain may transduce a signal to the interior of the host cell, eliciting an effector function, e.g., engulfment, phagolysosome maturation, or secretion of anti-inflammatory and/or immunosuppressive cytokines. An effector domain may directly promote a cellular response when it contains one or more signaling domains or motifs. In other embodiments, an effector domain will indirectly promote a cellular response by associating with one or more other proteins that directly promote a cellular response.

An “engulfment signaling domain” refers to an intracellular effector domain, which, upon binding of the target molecule (e.g., a neurodegenerative disease antigen) targeted by the extracellular domain of a CER expressed by a host cell, activates one or more signaling pathways in the host cell resulting in engulfment, including, in specific embodiments, cytoskeletal rearrangement of the host cell and internalization of the target cell, protein, peptide, prion, or particle associated with the neurodegenerative disease antigen. In certain embodiments, an engulfment signaling domain activates one or more signaling pathways resulting in phagocytosis of the target cell, prion, protein, peptide, or particle. In certain embodiments, an engulfment signaling domain comprises a homeostatic engulfment signaling domain. In further embodiments, an engulfment signaling domain comprises a primary homeostatic engulfment signaling domain and a secondary engulfment signaling domain, which may be homeostatic or non-homeostatic.

The term “homeostatic engulfment signaling domain” refers to an effector domain that (i) stimulates engulfment of the targeted cell, microbe, protein, or particle (ii) is derived from an endogenous receptor or signaling molecule that typically does not stimulate an inflammatory or immunogenic response. In some embodiments, a homeostatic engulfment signaling domain stimulates host cell secretion of anti-inflammatory and/or immunosuppressive cytokines, such as, for example, TGF-β and IL-10. In certain embodiments, stimulation of homeostatic engulfment signaling dampens, attenuates, or resolves inflammation in the local tissue milieu. A homeostatic engulfment signaling domain can also be referred to as a “non-inflammatory” engulfment signaling domain or a “non-immunogenic” engulfment signaling domain.

“Junction amino acids” or “junction amino acid residues” refer to one or more (e.g., about 2-20) amino acid residues between two adjacent motifs, regions or domains of a polypeptide. Junction amino acids may result from the construct design of a chimeric protein (e.g., amino acid residues resulting from the use of a restriction enzyme site during the construction of a nucleic acid molecule encoding a fusion protein).

A “disease” is a state of health of a subject wherein the subject cannot maintain homeostasis, and wherein, if the disease is not ameliorated, then the subject's health continues to deteriorate. In contrast, a “disorder” or “undesirable condition” in a subject is a state of health in which the subject is able to maintain homeostasis, but in which the subject's state of health is less favorable than it would be in the absence of the disorder or undesirable condition. Left untreated, a disorder or undesirable condition does not necessarily result in a further decrease in the subject's state of health.

A “neurodegenerative disease” or “neurodegenerative disorder” refers to any medical condition resulting from the progressive loss of structure or function of neurons, including neuronal death. A neurodegenerative disease may affect the normal function of the central nervous system (CNS), including the brain and spinal cord, or peripheral nervous system (PNS), including the nerves and ganglia outside the brain and spinal cord. A neurodegenerative disease may be caused by a multitude of factors, including genetic mutations and/or environmental exposure (e.g., toxins, chemicals, viruses). Exemplary neurodegenerative diseases include Lewy body disease, post-poliomyelitis syndrome, Shy-Draeger syndrome, olivopontocerebellar atrophy, Parkinson's disease, multiple system atrophy, striatonigral degeneration, frontotemporal lobar degeneration with ubiquitinated inclusions (FLTD-U), tauopathies (including, but not limited to, Alzheimer's disease and supranuclear palsy), prion diseases (also known as transmissible spongiform encephalopathies, including, but not limited to, bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease including Amyotrophic lateral sclerosis (Lou Gherig's disease), nervous system heredodegenerative disorders including, but not limited to, Canavan disease, Huntington's disease, neuronal ceroid-lipofuscinosis, Alexander disease, Tourette's syndrome, Menkes kinky hair syndrome, Cockayne syndrome, Halervorden-Spatz syndrome, lafora disease, hepatolenticular degeneration, Lesch-Nyhan syndrome, and Unverricht-Lundborg syndrome), dementia (including, but not limited to, Pick's disease, and spinocerebellar ataxia).

A “neurodegenerative disease antigen” refers to an antigen that is expressed in the CNS, including the brain, or PNS and can be targeted with an antibody, receptor, ligand, polypeptide, or small molecule. In certain embodiments, neurodegenerative disease antigen is a protein or peptide that is overexpressed or inappropriately expressed in the CNS or PNS. A neurodegenerative disease antigen may be an intracellular protein or peptide (e.g., cytoplasmic, within inclusion bodies), a protein or peptide expressed on the surface of a cell (e.g., neuron), or an extracellular protein or peptide. A neurodegenerative disease antigen may be an unfolded protein or peptide, a protein or peptide in its native conformation (correctly folded), or a misfolded protein or peptide. A neurodegenerative disease antigen may be a protein/or peptide monomer, oligomer, fibril, or aggregate. In certain embodiments, a neurodegenerative disease antigen is a prion or prion protein (PrP). Examples of neurodegenerative disease antigens include antigens from beta-secretase 1 (BACE1), amyloid-β, epidermal growth factor receptor (EGFR), human epidermal growth factor receptor 2 (HER2), Tau, apolipoprotein E4 (ApoE4), ataxin-2, alpha-synuclein, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6.

As used herein, the term “amyloid beta” or “beta-amyloid” or “Abeta,” “amyloidp” or “Aβ,” used interchangeably herein, refer to a fragment of amyloid precursor protein (APP) that is produced upon cleavage of APP by β-secretase 1 (“BACE1”) , as well as modifications, fragments and any functional equivalents thereof, including, but not limited to, Aβ1-40 peptide, and Aβ1-42 peptide. AP may be a monomer, or may associate to form oligomers or fibril structures. AP fibrils may aggregate into amyloid plaques, e.g., such as those found in brains of Alzheimer's disease patients.

A “particle” refers to a fragment of a cell or a small object of at least 10 nm and up to 50 μm in diameter and that is derived from a living cell or organism. A particle can be a viral particle, prion particle, protein particle, small mineral particle, cellular debris.

As used herein, the term “engulfment” refers to a receptor-mediated process wherein endogenous or exogenous cells, prions, extracellular proteins or peptides (e.g., native conformation, misfolded, oligomers, fibrils, or aggregates), or particles greater than 10 nm in diameter are internalized by a phagocyte or host cell of the present disclosure. Engulfment is typically composed of multiple steps: (1) tethering of the target cell, prion, protein, peptide, or particle via binding of an engulfment receptor to a neurodegenerative disease antigen directly or indirectly (via a bridging molecule) on the target cell, prion, protein, peptide, or particle; and (2) internalization or engulfment of the whole target cell, protein, peptide, or particle, or a portion of the whole target cell, protein, peptide, or particle. In certain embodiments, internalization may occur via cytoskeletal rearrangement of a phagocyte or host cell to form a phagosome, a membrane-bound compartment containing the internalized target. Engulfment may further include maturation of the phagosome, wherein the phagosome becomes increasingly acidic and fuses with lysosomes (to form a phagolysosome), whereupon the engulfed target is degraded (e.g., “phagocytosis”). Alternatively, phagosome-lysosome fusion may not be observed in engulfment. In yet another embodiment, a phagosome may regurgitate or discharge its contents to the extracellular environment before complete degradation. In some embodiments, engulfment refers to phagocytosis. In some embodiments, engulfment includes tethering of the target cell, prion, protein, peptide or particle by the phagocyte or host cell of the present disclosure, but not internalization. In some embodiments, engulfment includes tethering of the target cell, prion, protein, peptide, or particle by the phagocyte or host cell of the present disclosure and internalization of part of the target cell, prion, protein, peptide, or particle.

As used herein, the term “phagocytosis” refers to an engulfment process of cells, extracellular protein or peptide (e.g., native conformation, misfolded, oligomers, fibrils, or aggregates), or large particles (≥0.5 ≥m) wherein tethering of a target cell, protein, peptide, or particle, engulfment of the target cell, protein, peptide or particle, and degradation of the internalized target cell, protein, peptide, or particle occurs. In certain embodiments, phagocytosis comprises formation of a phagosome that encompasses the internalized target cell, protein, peptide, or particle and phagosome fusion with a lysosome to form a phagolysosome, wherein the contents therein are degraded. In certain embodiments, during phagocytosis, following binding of a CER expressed on a phagocyte or a host cell of the present disclosure to a neurodegenerative disease antigen, such as an antigen expressed by a target cell or a protein, peptide, or particle associated with neurodegenerative disease, a phagocytic synapse is formed; an actin-rich phagocytic cup is generated at the phagocytic synapse; phagocytic arms are extended around the target cell, protein, peptide, or particle through cytoskeletal rearrangements; and ultimately, the target cell, protein, peptide, or particle is pulled into the phagocyte or host cell through force generated by motor proteins. As used herein, “phagocytosis” includes the process of “efferocytosis,” which specifically refers to the phagocytosis of apoptotic or necrotic cells in a non-inflammatory manner.

“Nucleic acid molecule” and “polynucleotide” can be in the form of RNA or DNA, which includes cDNA, genomic DNA, and synthetic DNA. A nucleic acid molecule may be composed of naturally occurring nucleotides (such as deoxyribonucleotides and ribonucleotides), analogs of naturally occurring nucleotides (e.g., a-enantiomeric forms of naturally occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. A nucleic acid molecule may be double stranded or single stranded, and if single stranded, may be the coding strand or non-coding (anti-sense strand). A coding molecule may have a coding sequence identical to a coding sequence known in the art or may have a different coding sequence, which, as the result of the redundancy or degeneracy of the genetic code, or by splicing, can encode the same polypeptide.

“Encoding” refers to the inherent property of specific polynucleotide sequences, such as DNA, cDNA, and mRNA sequences, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a polynucleotide encodes a protein if transcription and translation of mRNA corresponding to that polynucleotide produces the protein in a cell or other biological system. Both a coding strand and a non-coding strand can be referred to as encoding a protein or other product of the polynucleotide. Unless otherwise specified, a “nucleotide sequence encoding an amino acid sequence” includes all nucleotide sequences that are degenerate versions of each other and that encode the same amino acid sequence.

As used herein, the term “endogenous” or “native” refers to a gene, protein, compound, molecule or activity that is normally present in a host or host cell, including naturally occurring variants of the gene, protein, compound, molecule, or activity.

As used herein, “homologous” or “homolog” refers to a molecule or activity from a host cell that is related by ancestry to a second gene or activity, e.g., from the same host cell, from a different host cell, from a different organism, from a different strain, from a different species. For example, a heterologous molecule or heterologous gene encoding the molecule may be homologous to a native host cell molecule or gene that encodes the molecule, respectively, and may optionally have an altered structure, sequence, expression level or any combination thereof.

As used herein, “heterologous” nucleic acid molecule, construct or sequence refers to a nucleic acid molecule or portion of a nucleic acid molecule that is not native to a host cell, but can be homologous to a nucleic acid molecule or portion of a nucleic acid molecule from the host cell. The source of the heterologous nucleic acid molecule, construct or sequence can be from a different genus or species. In some embodiments, the heterologous nucleic acid molecules are not naturally occurring. In certain embodiments, a heterologous nucleic acid molecule is added (i.e., not endogenous or native) into a host cell or host genome by, for example, conjugation, transformation, transfection, transduction, electroporation, or the like, wherein the added molecule can integrate into the host cell genome or exist as extra-chromosomal genetic material (e.g., as a plasmid or other form of self-replicating vector), and can be present in multiple copies. In addition, “heterologous” refers to a non-native enzyme, protein or other activity encoded by a non-endogenous nucleic acid molecule introduced into the host cell, even if the host cell encodes a homologous protein or activity.

As used herein, the term “engineered,” “recombinant,” “modified” or “non-natural” refers to an organism, microorganism, cell, nucleic acid molecule, or vector that has been modified by introduction of an heterologous nucleic acid molecule, or refers to a cell or microorganism that has been genetically engineered by human intervention—that is, modified by introduction of a heterologous nucleic acid molecule, or refers to a cell or microorganism that has been altered such that expression of an endogenous nucleic acid molecule or gene is controlled, deregulated or constitutive, where such alterations or modifications can be introduced by genetic engineering. Human-generated genetic alterations can include, for example, modifications introducing nucleic acid molecules (which may include an expression control element, such as a promoter) encoding one or more proteins, chimeric receptors, or enzymes, or other nucleic acid molecule additions, deletions, substitutions, or other functional disruption of or addition to a cell's genetic material. Exemplary modifications include those in coding regions or functional fragments thereof heterologous or homologous polypeptides from a reference or parent molecule. Additional exemplary modifications include, for example, modifications in non-coding regulatory regions in which the modifications alter expression of a gene or operon.

The term “overexpressed” or “overexpression” of an antigen refers to an abnormally high level of antigen expression in a cell. Overexpressed antigen or overexpression of antigen is often associated with a disease state, such as in neurodegenerative diseases within a specific tissue or organ of the CNS or PNS of a subject. Neurodegenerative diseases characterized by overexpression of a neurodegenerative disease antigen can be determined by standard assays known in the art.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “mature polypeptide” or “mature protein” refers to a protein or polypeptide that is secreted or localized in the cell membrane or inside certain cell organelles (e.g., the endoplasmic reticulum, golgi, or endosome) and does not include an N-terminal signal peptide.

A “signal peptide,” also referred to as “signal sequence,” “leader sequence,” “leader peptide,” “localization signal” or “localization sequence,” is a short peptide (usually 15-30 amino acids in length) present at the N-terminus of newly synthesized proteins that are destined for the secretory pathway. A signal peptide typically comprises a short stretch of hydrophilic, positively charged amino acids at the N-terminus, a central hydrophobic domain of 5-15 residues, and a C-terminal region with a cleavage site for a signal peptidase. In eukaryotes, a signal peptide prompts translocation of the newly synthesized protein to the endoplasmic reticulum where it is cleaved by the signal peptidase, creating a mature protein that then proceeds to its appropriate destination.

The “percent identity” between two or more nucleic acid or amino acid sequences is a function of the number of identical positions shared by the sequences (i.e., % identity=number of identical positions/total number of positions×100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST). A “conservative substitution” is recognized in the art as a substitution of one amino acid for another amino acid that has similar properties. Exemplary conservative substitutions are well known in the art (see, e.g., WO 97/09433, page 10, published Mar. 13, 1997; Lehninger, Biochemistry, Second Edition; Worth Publishers, Inc. NY:NY (1975), pp.71-'7′7; Lewin, Genes IV, Oxford University Press, NY and Cell Press, Cambridge, Mass. (1990), p. 8).

The term “chimeric” refers to any nucleic acid molecule or protein that is not endogenous and comprises a combination of sequences joined or linked together that are not naturally found joined or linked together in nature. For example, a chimeric nucleic acid molecule may comprise nucleic acids encoding various domains from multiple different genes. In another example, a chimeric nucleic acid molecule may comprise regulatory sequences and coding sequences that are derived from different sources, or regulatory sequences and coding sequences that are derived from the same source but arranged in a manner different than that found in nature.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The phrase “under transcriptional control” or “operatively linked” as used herein means that a promoter is in the correct location and orientation in relation to a polynucleotide to control the initiation of transcription by RNA polymerase and expression of the polynucleotide.

A “vector” is a nucleic acid molecule that is capable of transporting another nucleic acid. Vectors may be, for example, plasmids, cosmids, viruses, or phage. The term should also be construed to include non-plasmid and non-viral compounds which facilitate transfer of nucleic acid into cells. An “expression vector” is a vector that is capable of directing the expression of a protein encoded by one or more genes carried by the vector when it is present in the appropriate environment.

In certain embodiments, the vector is a viral vector. Examples of viral vectors include, but are not limited to, adenovirus vectors, adeno-associated virus vectors, retrovirus vectors, gammaretrovirus vectors, and lentivirus vectors. “Retroviruses” are viruses having an RNA genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses. “Lentivirus” refers to a genus of retroviruses that are capable of infecting dividing and non-dividing cells. Examples of lentiviruses include, but are not limited to HIV (human immunodeficiency virus, including HIV type 1 and HIV type 2, equine infectious anemia virus, feline immunodeficiency virus (FIV), bovine immune deficiency virus (BIV), and simian immunodeficiency virus (SIV).

In other embodiments, the vector is a non-viral vector. Examples of non-viral vectors include lipid-based DNA vectors, modified mRNA (modRNA), self-amplifying mRNA, closed-ended linear duplex (CELiD) DNA, and transposon-mediated gene transfer (PiggyBac, Sleeping Beauty). Where a non-viral delivery system is used, the delivery vehicle can be a liposome. Lipid formulations can be used to introduce nucleic acids into a host cell in vitro, ex vivo, or in vivo. The nucleic acid may be encapsulated in the interior of a liposome, interspersed within the lipid bilayer of a liposome, attached to a liposome via a linking molecule that is associated with both the liposome and the nucleic acid, contained or complexed with a micelle, or otherwise associated with a lipid.

The term “subject,” “patient” and “individual” are used interchangeably herein and are intended to include living organisms in which an immune response can be elicited (e.g., mammals). Examples of subjects include humans, primates, cows, horses, sheep, dogs, cats, mice, rats, rabbits, guinea pigs, pigs, and transgenic species thereof.

The term “immune system cell” or “immune cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Exemplary immune system cells include a CD4+ T cell, a CD8+ T cell, a CD4- CD8- double negative T cell, a γδ T cell, a regulatory T cell, a natural killer cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

The term “T cells” refers to cells of T cell lineage. “Cells of T cell lineage” refers to cells that show at least one phenotypic characteristic of a T cell or a precursor or progenitor thereof that distinguishes the cells from other lymphoid cells, and cells of the erythroid or myeloid lineages. Such phenotypic characteristics can include expression of one or more proteins specific for T cells (e.g. , CD3⁺, CD4⁺, CD8⁺), or a physiological, morphological, functional, or immunological feature specific for a T cell. For example, cells of the T cell lineage may be progenitor or precursor cells committed to the T cell lineage; CD25⁺ immature and inactivated T cells; cells that have undergone CD4 or CD8 linage commitment; thymocyte progenitor cells that are CD4⁺CD8⁺ double positive; single positive CD4⁺ or CD8⁺; TCRαβ or TCR γδ; or mature and functional or activated T cells. The term “T cells” encompasses naive T cells (CD45 RA+, CCR7+, CD62L+, CD27+, CD45RO−), central memory T cells (CD45RO⁺, CD62L⁺, CD8⁺), effector memory T cells (CD45RA+, CD45RO−, CCR7−, CD62L−, CD27−), mucosal-associated invariant T (MAIT) cells, Tregs, natural killer T cells, and tissue resident T cells.

The term “B cells” refers to cells of the B cell lineage. “Cells of B cell lineage” refers to cells that show at least one phenotypic characteristic of a B cell or a precursor or progenitor thereof that distinguishes the cells from other lymphoid cells, and cells of the erythroid or myeloid lineages. Such phenotypic characteristics can include expression of one or more proteins specific for B cells (e.g. , CD19⁺, CD72+, CD24+, CD20⁺), or a physiological, morphological, functional, or immunological feature specific for a B cell. For example, cells of the B cell lineage may be progenitor or precursor cells committed to the B cell lineage (e.g., pre-pro-B cells, pro-B cells, and pre-B cells); immature and inactivated B cells or mature and functional or activated B cells. Thus, “B cells” encompass naïe B cells, plasma cells, regulatory B cells, marginal zone B cells, follicular B cells, lymphoplasmacytoid cells, plasmablast cells, and memory B cells (e.g., CD27⁺, IgD⁻).

“Adoptive cellular immunotherapy” or “adoptive immunotherapy” refers to the administration of naturally occurring or genetically engineered, disease antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

“Autologous” refers to a graft (e.g., organ, tissue, cells) derived from the same subject to which it is later to be re-introduced.

“Allogeneic” refers to a graft derived from a different subject of the same species.

A “therapeutically effective amount” or “effective amount” of a chimeric protein or cell expressing a chimeric protein of this disclosure (e.g., a CER or a cell expressing a CER) refers to that amount of protein or cells sufficient to result in amelioration of one or more symptoms of the disease, disorder, or undesired condition being treated. When referring to an individual active ingredient or a cell expressing a single active ingredient, administered alone, a therapeutically effective dose refers to the effects of that ingredient or cell expressing that ingredient alone. When referring to a combination, a therapeutically effective dose refers to the combined amounts of active ingredients or combined adjunctive active ingredient with a cell expressing an active ingredient that results in a therapeutic effect, whether administered serially or simultaneously.

“Treat” or “treatment” or “ameliorate” refers to medical management of a disease, disorder, or undesired condition of a subject. In general, an appropriate dose or treatment regimen comprising a host cell expressing a CER of this disclosure is administered in an amount sufficient to elicit a therapeutic or prophylactic benefit. Therapeutic or prophylactic/preventive benefit includes improved clinical outcome; lessening or alleviation of symptoms associated with a disease, disorder, or undesired condition; decreased occurrence of symptoms; improved quality of life; longer disease-free status; diminishment of extent of disease, disorder, or undesired condition; stabilization of disease state; delay of disease progression; remission; survival; prolonged survival; or any combination thereof.

Additional definitions are provided throughout the present disclosure.

### Chimeric Engulfment Receptors (CERs)

In one aspect, the present disclosure provides a CER comprising a single chain chimeric protein, the single chain chimeric protein comprising: an extracellular domain comprising a binding domain that specifically binds to a neurodegenerative disease antigen; an engulfment signaling domain comprising a homeostatic engulfment signaling domain; and a transmembrane domain positioned between and connecting the extracellular domain and engulfment signaling domain.

Progressive accumulation of specific protein aggregates is a feature of many neurodegenerative diseases, including for example Alzheimer's disease, Parkinson's disease, fronto-temporal dementia, Huntington's disease, and Creutzfeldt-Jakob disease. Toxic protein accumulation may occur in different parts of the brain and can be in the nucleus, cytoplasm, or extracellular space. The CERs of the present disclosure may be used in adoptive immunotherapy compositions for promoting clearance of cells expressing a neurodegenerative disease antigen, of proteins, peptides, or particles associated with neurodegenerative disease, or extracellular protein aggregates. In certain aspects, the present disclosure provides CERs that may prevent, inhibit, or reduce the transfer of these aggregates to neighboring neurons, thereby preventing, inhibiting, or reducing aggregate spreading.

The engulfment signaling domain can include one or more effector (also referred to as “signaling”) domains that drive engulfment of the targeted cell, proteins, peptides, particles, or protein aggregates. Signaling by the engulfment signaling domain is triggered by binding of the extracellular domain to the targeted neurodegenerative disease antigen. In certain embodiments, an engulfment signaling domain comprises a homeostatic engulfment signaling domain. A homeostatic engulfment signaling domain may promote a non-immunogenic microenvironment or anti-inflammatory and/or immunosuppressive cytokines in the CNS or PNS, where an inflammatory response may be undesirable. In further embodiments, the engulfment signaling domain comprises a primary homeostatic engulfment signaling domain and a secondary engulfment signaling domain. The secondary engulfment signaling domain may be a homeostatic engulfment signaling domain or a non-homeostatic engulfment signaling domain.

Component parts of the fusion proteins of the present disclosure are further described in detail herein.

### Extracellular Domain

As described herein, a CER comprises an extracellular domain specific to a neurodegenerative disease target antigen. In certain embodiments, the extracellular domain comprises a binding domain that specifically binds a target molecule, i.e., a neurodegenerative disease antigen. Binding of a target molecule by the binding domain may block the interaction between the target molecule (e.g., a receptor or a ligand) and another molecule and, for example, interfere with, reduce or eliminate certain functions of the target molecule (e.g., signal transduction). In some embodiments, the binding of a target molecule may induce certain biological pathways or identify the target molecule or cell expressing the target molecule for elimination.

A binding domain suitable for use in a CER of the present disclosure may be any polypeptide or peptide that specifically binds a target molecule of interest, e.g., a neurodegenerative disease antigen. Sources of binding domains include extracellular domains of receptors, ligands for cell surface receptors or molecules, and antibodies or antigen binding portions, such as antibody variable regions from various species. For example a binding domain may comprise a, sFv, scFv, Fab, scFv-based grababody, VH domain, VL domain, single domain camelid antibody (VHH), or domain antibody. A binding domain may be derived from a human, primate, rodent, avian, or ovine. Additional sources of binding domains include variable regions of antibodies from other species, such as camelid (from camels, dromedaries, or llamas; Ghahroudi et al., FEBS Lett. 414:521, 1997; Vincke et al., J. Biol. Chem. 284:3273, 2009; Hamers-Casterman et al., Nature 363:446, 1993 and Nguyen et al., J. Mol. Biol. 275:413, 1998), nurse sharks (Roux et al., Proc. Nat'l. Acad. Sci. (USA) 95:11804, 1998), spotted ratfish (Nguyen et al., Immunogen. 54:39, 2002), or lamprey (Herrin et al., Proc. Nat'l. Acad. Sci. (USA) 105:2040, 2008 and Alder et al. Nat. Immunol. 9:319, 2008). These antibodies can form antigen-binding regions using only a heavy chain variable region, i.e., these functional antibodies are homodimers of heavy chains only (referred to as “heavy chain antibodies”) (Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008). In certain embodiments, a binding domain is murine, chimeric, human, or humanized.

Examples of neurodegenerative disease antigens include amyloid-β peptide, Tau, beta-secretase, apoliprotein E4 (ApoE4), alpha-synuclein, leucine rich repeat kinase 2 (LRRK2), presenlin 1, presenilin 2, parkin, gamma secretase, amyloid precursor protein (APP), beta-secretase (BACE1), mutated huntingtin protein (mHTT), Cu,Zn-superoxide dismutase-1 (SOD1), ataxin-2, TAR DNA-binding protein 43 (TDP-43), p75 neurotrophin receptor (p75NTR), semaphorin 4D (SEMA4D), protease-resistant prion protein (Prp^{res}) or pathogenic prion protein (PrP^{Sc}).

In certain embodiments, the binding domain comprises an antibody or antigen binding fragment thereof, such as a single chain Fv fragment (scFv) that comprises VH and VL regions, specific for a neurodegenerative disease antigen. In certain embodiments, the antibody or antigen binding fragment is chimeric, human, or humanized. In further embodiments, the V_{H} and V_{L} regions are human or humanized.

In certain embodiments, the binding domain comprises a scFv specific for a neurodegenerative disease antigen. In certain embodiments, a neurodegenerative disease antigen is, for example amyloid-β peptide, Tau, beta-secretase, apolipoprotein E4 (ApoE4), alpha-synuclein, leucine rich repeat kinase 2 (LRRK2), presenlin 1, presenilin 2, parkin, gamma secretase, amyloid precursor protein (APP), beta-secretase (BACE1), mutated huntingtin protein (mHTT), Cu,Zn-superoxide dismutase-1 (SOD1), TAR DNA-binding protein 43 (TDP-43), p75 neurotrophin receptor (p75NTR), semaphorin 4D (SEMA4D), ataxin-2, protease-resistant prion protein (PrP^{res}), or pathogenic prion protein (PrP^{Sc}), and exemplary V_{H} and V_{L} regions include the segments of anti-amyloid-β, anti-Tau, anti-beta-secretase, anti-ApoE4, anti-alpha-synuclein, anti-LRRK2, anti-presenlin 1, anti-presenilin 2, anti-parkin, anti-gamma secretase, anti-amyloid precursor protein, anti-APP, anti-BACE1, anti-mHTT, anti-SOD1, anti-TDP-43, anti-p75NTR, anti-SEMA4D, anti-ataxin-2, anti-PrP^{res} or anti-PrP^{Sc} specific monoclonal antibodies, respectively.

In further embodiments, the binding domain comprises a Fab specific for a neurodegenerative disease antigen. In certain embodiments, a neurodegenerative disease antigen is, for example amyloid-βpeptide, Tau, beta-secretase, apolipoprotein E4 (ApoE4), alpha-synuclein, leucine rich repeat kinase 2 (LRRK2), presenlin 1, presenilin 2, parkin, gamma secretase, amyloid precursor protein (APP), beta-secretase (BACE1), huntingtin prion protein (PrP), Cu,Zn-superoxide dismutase-1 (SOD1), TAR DNA-binding protein 43 (TDP-43), p75 neurotrophin receptor (p75NTR), SEMA4D, ataxin-2, PrP^{res}, or PrP^{Sc} and Fab regions include portions of anti-amyloid-β, anti-Tau, anti-beta-secretase, anti-ApoE4, anti-alpha-synuclein, anti-LRRK2, anti-presenlin 1, anti-presenilin 2, anti-parkin, anti-gamma secretase, anti-amyloid precursor protein, anti-APP, anti-BACE1, anti-mHTT, anti-SOD1, anti-TDP-43, anti-p75NTR, anti-SEMA4D, anti-ataxin-2, anti-PrP^{res} or anti-PrP^{Sc} specific monoclonal antibodies, respectively.

In certain embodiments, the binding domain comprises a scFv derived from BIIB037 antibody (aducanumab), which is a human IgG1 monoclonal antibody specific for aggregated amyloid-β. An exemplary scFv derived from BIIB037 antibody comprises an amino acid sequence of SEQ ID NO:2.

In certain embodiments, the binding domain comprises a scFv derived from bapineuzumab, which is a humanized IgG1 antibody that binds to soluble monomers, fibrils, and plaques of amyloid-β (see, U.S. Patent Publication No. 2008/0292625).

In certain embodiments, the binding domain comprises a scFv derived from crenezumab, which is a humanized antibody that binds to amyloid-β monomers, oligomers, fibrils, and plaques (see, U.S. Pat. 7,892,544).

In certain embodiments, the binding domain comprises a scFv derived from solanezumab, which is a humanized IgG1 antibody that binds to soluble amyloid-β monomers (see, U.S. Pat. No. 7,195,761).

In certain embodiments, the binding domain comprises a scFv derived from ponezumab, a humanized IgG2A antibody that binds to amyloid-β.

In certain embodiments, the binding domain comprises a scFv derived from gantenerumab, a human IgG1 antibody that binds to amyloid-β.

In certain embodiments, the binding domain comprises a scFv derived from BAN-2401 antibody, a humanized IgG1 antibody that binds to amyloid-β protofibrils (see, U.S. Pat. 8,025,878).

In certain embodiments, the binding domain comprises a scFv derived from ABBV-8E12 (also known as C2N-8E12) antibody, a humanized IgG4 antibody that binds Tau (see, U.S. Patent Publication No. 2017/0058024).

In certain embodiments, the binding domain comprises a scFv derived from BMS-986168 (also known as BIIB092) antibody, a humanized antibody that binds extracellular Tau.

In certain embodiments, the binding domain comprises a scFv derived from BIIB076 antibody, a human pan-Tau antibody.

In certain embodiments, the binding domain comprises a scFv derived from R07105705 antibody, a human pan-Tau antibody.

In certain embodiments, the binding domain comprises a scFv derived from RG7345 antibody, which is a human antibody that binds to Tau/pS422.

In certain embodiments, the binding domain comprises a scFv derived from PRX002 antibody, which is a humanized IgG1 antibody that binds to a-synuclein (see, U.S. Pat. No. 7,910,333).

In certain embodiments, the binding domain comprises a scFv derived from BIIB054 antibody, which is a human antibody that binds to aggregated α-synuclein.

In certain embodiments, the binding domain comprises a scFv derived from 12F4 antibody, which is a human antibody that binds to a-synuclein (see, U.S. Pat. No. 8,940,276).

In certain embodiments, the binding domain comprises a scFv derived from VX15, an antibody that binds to semaphorin 4D (see, U.S. Pat. No. 8,496,938).

A target molecule, which is specifically bound by an extracellular domain of a CER of the present disclosure, may be found on or in association with a cell of interest (“target cell”), or a non-cellular component, such as a prion, misfolded protein, protein aggregate, or protein fibril. Exemplary target cells include neurons. Neurons, also known as nerve cells, make up the CNS and PNS. Exemplary neurons include sensory neurons, motor neurons, and interneurons. In certain embodiments, a neuron is a brain neuron. Brain neurons include, but are not limited to, Purkinje cells, granule cells, basket cells, stellate cells, Golgi cells, pyramidal cells, chandelier cells, candelabrum cells, unipolar brush cells, and spindle neurons.

In certain embodiments, the extracellular domain optionally comprises an extracellular, non-signaling spacer or linker domain. Where included, such a spacer or linker domain may position the binding domain away from the host cell surface to further enable proper cell to cell/aggregate/protein/or particle contact, binding, and activation. An extracellular spacer domain is generally located between the extracellular binding domain and the transmembrane domain of the CER. The length of the extracellular spacer may be varied to optimize target molecule binding based on the selected target molecule, selected binding epitope, binding domain size and affinity (see, e.g., Guest et al., J. Immunother. 28:203-11, 2005; PCT Publication No. WO 2014/031687). In certain embodiments, an extracellular spacer domain is an immunoglobulin hinge region (e.g., IgG1, IgG2, IgG3, IgG4, IgA, IgD). An immunoglobulin hinge region may be a wild type immunoglobulin hinge region or an altered wild type immunoglobulin hinge region. An altered IgG₄ hinge region is described in PCT Publication No. WO 2014/031687, which hinge region is incorporated herein by reference in its entirety. In a particular embodiment, an extracellular spacer domain comprises a modified IgG₄ hinge region having an amino acid sequence of ESKYGPPCPPCP (SEQ ID NO:3).

Other examples of hinge regions that may be used in the CERs described herein include the hinge region from the extracellular regions of type 1 membrane proteins, such as CD8a, CD4, CD28 and CD7, which may be wild-type or variants thereof. In further embodiments, an extracellular spacer domain comprises all or a portion of an immunoglobulin Fc domain selected from: a CH1 domain, a CH2 domain, a CH3 domain, or combinations thereof (see, e.g., PCT Publication WO2014/031687, which spacers are incorporated herein by reference in their entirety). In yet further embodiments, an extracellular spacer domain may comprise a stalk region of a type II C-lectin (the extracellular domain located between the C-type lectin domain and the transmembrane domain). Type II C-lectins include CD23, CD69, CD72, CD94, NKG2A, and NKG2D. In yet further embodiments, an extracellular spacer domain may be derived from a toll-like receptor (TLR) juxtamembrane domain. A TLR juxtamembrane domain comprises acidic amino acids lying between the leucine rich repeats (LRRs) and the transmembrane domain of a TLR. In certain embodiments, a TLR juxtamembrane domain is a TLR1, TLR2, TLR3, TLR4, TLR5, TLR6, TLR7, TLR8, or TLR9 juxtamembrane domain. An exemplary TLR juxtamembrane domain is a TLR4 juxtamembrane domain comprising an amino acid sequence of SEQ ID NO:4.

### Engulfment Signaling Domain

The engulfment signaling domain of a CER is an intracellular effector domain and is capable of transmitting functional signals to a cell in response to binding of the extracellular domain of the CER to a target molecule. The engulfment signaling domain may be any portion of an engulfment signaling molecule that retains sufficient signaling activity. In some embodiments, a full length or full length intracellular component of an engulfment signaling molecule is used. In some embodiments, a truncated portion of an engulfment signaling molecule or intracellular component of an engulfment signaling molecule is used, provided that the truncated portion retains sufficient signal transduction activity. In further embodiments, an engulfment signaling domain is a variant of an entire or truncated portion of an engulfment signaling molecule, provided that the variant retains sufficient signal transduction activity (i.e., is a functional variant).

In certain embodiments, an engulfment signaling domain comprises a homeostatic engulfment signaling domain. Exemplary homeostatic engulfment signaling domains include a MRC1 signaling domain, a MERTK signaling domain, a Tyro3 signaling domain, an Axl signaling domain, or an ELMO signaling domain. In some embodiments, the homeostatic engulfment signaling domain comprises a sequence that has at least about 75%, 80%, 85%, 90%, 91%, 92%, 93%, 94%, 95%, 96%, 97%, 98%, 99%, 99.5%, or 100% identity to an MRC1 signaling domain comprising an amino acid sequence of SEQ ID NO:5, a MERTK signaling domain comprising an amino acid sequence of SEQ ID NO:6, a Tyro3 signaling domain comprising an amino acid sequence of SEQ ID NO:7, an Axl signaling domain comprising an amino acid sequence of SEQ ID NO:8, or an ELMO signaling domain comprising an amino acid sequence of SEQ ID NO:9. In some embodiments, the homeostatic engulfment signaling domain is an MRC1 signaling domain comprising or consisting of an amino acid sequence of SEQ ID NO:5, a MERTK signaling domain comprising or consisting of an amino acid sequence of SEQ ID NO:6, a Tyro3 signaling domain comprising or consisting of an amino acid sequence of SEQ ID NO:7, an Axl signaling domain comprising or consisting of an amino acid sequence of SEQ ID NO:8, or an ELMO signaling domain comprising or consisting of an amino acid sequence of SEQ ID NO:9.

A truncated homeostatic engulfment signaling domain may be truncated at its N-terminus, its C-terminus, at both the N-terminus and C-terminus. In certain embodiments, the MRC1 homeostatic engulfment signaling domain is truncated 1, 2, 3, 4, 5, or more amino acids at its N-terminus corresponding to the amino acid sequence of SEQ ID NO:5; the MERTK homeostatic engulfment signaling domain is truncated 1, 2, 3, 4, 5, or more amino acids at its N-terminus corresponding to the amino acid sequence of SEQ ID NO:6; the Tyro3 homeostatic engulfment signaling domain is truncated 1, 2, 3, 4, 5, or more amino acids at its N-terminus corresponding to the amino acid sequence of SEQ ID NO:7; the Axl homeostatic engulfment signaling domain is truncated 1, 2, 3, 4, 5, or more amino acids at its N-terminus corresponding to the amino acid sequence of SEQ ID NO:8; or the ELMO homeostatic engulfment signaling domain is truncated 1, 2, 3, 4, 5, or more amino acids at its N-terminus corresponding to the amino acid sequence of SEQ ID NO:9. In certain embodiments, the MRC1 homeostatic engulfment signaling domain is truncated 1, 2, 3, 4, 5, or more amino acids at its C-terminus corresponding to the amino acid sequence of SEQ ID NO:5; the MERTK homeostatic engulfment signaling domain is truncated 1, 2, 3, 4, 5, or more amino acids at its C-terminus corresponding to the amino acid sequence of SEQ ID NO:6; the Tyro3 homeostatic engulfment signaling domain is truncated 1, 2, 3, 4, 5, or more amino acids at its C-terminus corresponding to the amino acid sequence of SEQ ID NO:7; the Axl homeostatic engulfment signaling domain is truncated 1, 2, 3, 4, 5, or more amino acids at its C-terminus corresponding to the amino acid sequence of SEQ

ID NO:8; or the ELMO homeostatic engulfment signaling domain is truncated 1, 2, 3, 4, 5, or more amino acids at its C-terminus corresponding to the amino acid sequence of SEQ ID NO:9.

In further embodiments, the engulfment signaling domain can include more than one signaling domain. In certain such embodiments, the engulfment signaling domain comprises a primary homeostatic engulfment signaling domain and a secondary engulfment signaling domain. The primary engulfment signaling domain may be N-terminal to the secondary engulfment signaling domain or C-terminal to the secondary engulfment signaling domain. Exemplary secondary engulfment signaling domains include a MRC1 signaling domain, a MERTK signaling domain, a Tyro3 signaling domain, an Axl signaling domain, an ELMO signaling domain, a Traf6 signaling domain, a Syk signaling domain, a MyD88 signaling domain, a PI3K signaling domain, a FcR signaling domain (e.g., FcγR1, FcγR2A, FcγR2C, FcγR2B2 , FcγR3A , FcγR2C , FcγR3A , FcϵR1, or FcαR1 signaling domain), a B-cell activating factor receptor (BAFF-R) signaling domain, a DAP12 (also referred to as TYRO Protein Tyrosine Kinase Binding Protein (TYROBP)) signaling domain, an NFAT Activating Protein With ITAM Motif 1 (NFAM1) signaling domain, a CD79b signaling domain, a TLR signaling domain (e.g., TLR1, TLR2, TLR3, TLR4, TLR5, TLR6, TLR7, TLR8, or TLR9 signaling domain), a Traf2 signaling domain, or a Traf 3 signaling domain.

In some embodiments, the secondary engulfment signaling domain comprises a sequence that has at least about 75%, 80%, 85%, 90%, 91%, 92%, 93%, 94%, 95%, 96%, 97%, 98%, 99%, 99.5%, or 100% identity to a MRC1 signaling domain comprising an amino acid sequence of SEQ ID NO:5, a MERTK signaling domain comprising an amino acid sequence of SEQ ID NO:6, a Tyro3 signaling domain comprising an amino acid sequence of SEQ ID NO:7, an Axl signaling domain comprising an amino acid sequence of SEQ ID NO:8, an ELMO signaling domain comprising an amino acid sequence of SEQ ID NO:9, a Traf6 signaling domain comprising an amino acid sequence of SEQ ID NO:10, a Syk signaling domain comprising an amino acid sequence of SEQ ID NO:11, a MyD88 signaling domain comprising an amino acid sequence of SEQ ID NO:12, a FcϵRIγ signaling domain comprising an amino acid sequence of SEQ ID NO:14, a FcγR1 signaling domain comprising an amino acid sequence of SEQ ID NO:15, a FcγR2A signaling domain comprising an amino acid sequence of SEQ ID NO:16, a FcγR2C signaling domain comprising an amino acid sequence of SEQ ID NO:17, a FcγR3A signaling domain comprising an amino acid sequence of SEQ ID NO:18, a BAFF-R signaling domain comprising an amino acid sequence of SEQ ID NO:19, a DAP12 signaling domain comprising an amino acid sequence of SEQ ID NO:20, a NFAM1 signaling domain comprising an amino acid sequence of SEQ ID NO:21, a CD79b signaling domain comprising an amino acid sequence of SEQ ID NO:84, a TLR1 signaling domain comprising an amino acid sequence of SEQ ID NO:23, a TLR2 signaling domain comprising an amino acid sequence of SEQ ID NO:24, a TLR3 signaling domain comprising an amino acid sequence of SEQ ID NO:25, a TLR4 signaling domain comprising an amino acid sequence of SEQ ID NO:26, a TLR5 signaling domain comprising an amino acid sequence of SEQ ID NO:27, a TLR6 signaling domain comprising an amino acid sequence of SEQ ID NO:28, a TLR7 signaling domain comprising an amino acid sequence of SEQ ID NO:29, a TLR8 signaling domain comprising an amino acid sequence of SEQ ID NO:30, a TLR9 signaling domain comprising an amino acid sequence of SEQ ID NO:31, a Traf2 signaling domain comprising an amino acid sequence of SEQ ID NO:32, or a Traf3 signaling domain comprising an amino acid sequence of SEQ ID NO:33.

In some embodiments, the secondary engulfment signaling domain is an MRC1 signaling domain comprising or consisting of an amino acid sequence of SEQ ID NO:5, a MERTK signaling domain comprising or consisting of an amino acid sequence of SEQ ID NO:6, a Tyro3 signaling domain comprising or consisting of an amino acid sequence of SEQ ID NO:7, an Axl signaling domain comprising or consisting of an amino acid sequence of SEQ ID NO:8, or an ELMO signaling domain comprising or consisting of an amino acid sequence of SEQ ID NO:9, a Traf6 signaling domain comprising or consisting of an amino acid sequence of SEQ ID NO:10, a Syk signaling domain comprising or consisting of an amino acid sequence of SEQ ID NO:11, a MyD88 signaling domain comprising or consisting of an amino acid sequence of SEQ ID NO:12, a FcϵRIγ signaling domain comprising or consisting of an amino acid sequence of SEQ ID NO:14, a FcγR1 signaling domain comprising or consisting of an amino acid sequence of SEQ ID NO:15, a FcγR2A signaling domain comprising or consisting of an amino acid sequence of SEQ ID NO:16, a FcγR2C signaling domain comprising or consisting of an amino acid sequence of SEQ ID NO:17, a FcγR3A signaling domain comprising or consisting of an amino acid sequence of SEQ ID NO:18, a BAFF-R signaling domain comprising or consisting of an amino acid sequence of SEQ ID NO:19, a DAP-12 signaling domain comprising or consisting of an amino acid sequence of SEQ ID NO:20, a NFAM1 signaling domain comprising or consisting of an amino acid sequence of SEQ ID NO:21, a CD79b signaling domain comprising or consisting of an amino acid sequence of SEQ ID NO:84, a TLR1 signaling domain comprising or consisting of an amino acid sequence of SEQ ID NO:23, a TLR2 signaling domain comprising or consisting of an amino acid sequence of SEQ ID NO:24, a TLR3 signaling domain comprising or consisting of an amino acid sequence of SEQ ID NO:25, a TLR4 signaling domain comprising or consisting of an amino acid sequence of SEQ ID NO:26, a TLR5 signaling domain comprising or consisting of an amino acid sequence of SEQ ID NO:27, a TLR6, signaling domain comprising or consisting of an amino acid sequence of SEQ ID NO:28, a TLR7 signaling domain comprising or consisting of an amino acid sequence of SEQ ID NO:29, a TLR8, signaling domain comprising or consisting of an amino acid sequence of SEQ ID NO:30, a TLR9 signaling domain comprising or consisting of an amino acid sequence of SEQ ID NO:31, a Traf2 signaling domain comprising or consisting of an amino acid sequence of SEQ ID NO:32, or a Traf3 signaling domain comprising or consisting of an amino acid sequence of SEQ ID NO:33.

A truncated secondary engulfment signaling domain may be truncated at its N-terminus, its C-terminus, at both the N-terminus and C-terminus. In certain embodiments, the MRC1 engulfment signaling domain is truncated 1, 2, 3, 4, 5, or more amino acids at its N-terminus corresponding to the amino acid sequence of SEQ ID NO:5; the MERTK engulfment signaling domain is truncated 1, 2, 3, 4, 5, or more amino acids at its N-terminus corresponding to the amino acid sequence of SEQ ID NO:6; the Tyro3 engulfment signaling domain is truncated 1, 2, 3, 4, 5, or more amino acids at its N-terminus corresponding to the amino acid sequence of SEQ ID NO:7; the Axl engulfment signaling domain is truncated 1, 2, 3, 4, 5, or more amino acids at its N-terminus corresponding to the amino acid sequence of SEQ ID NO:8; the ELMO engulfment signaling domain is truncated 1, 2, 3, 4, 5, or more amino acids at its N-terminus corresponding to the amino acid sequence of SEQ ID NO:9; the Traf6 engulfment signaling domain is truncated 1, 2, 3, 4, 5, or more amino acids at its N-terminus corresponding to the amino acid sequence of SEQ ID NO:10; the Syk engulfment signaling domain is truncated 1, 2, 3, 4, 5, or more amino acids at its N-terminus corresponding to the amino acid sequence of SEQ ID NO:11; the MyD88 engulfment signaling domain is truncated 1, 2, 3, 4, 5, or more amino acids at its N-terminus corresponding to the amino acid sequence of SEQ ID NO:12; the FcϵRIγ engulfment signaling domain is truncated 1, 2, 3, 4, 5, or more amino acids at its N-terminus corresponding to the amino acid sequence of SEQ ID NO:14; the FcγR1 engulfment signaling domain is truncated 1, 2, 3, 4, 5, or more amino acids at its N-terminus corresponding to the amino acid sequence of SEQ ID NO:15; the FcγR2A engulfment signaling domain is truncated 1, 2, 3, 4, 5, or more amino acids at its N-terminus corresponding to the amino acid sequence of SEQ ID NO:16; the FcγR2C engulfment signaling domain is truncated 1, 2, 3, 4, 5, or more amino acids at its N-terminus corresponding to the amino acid sequence of SEQ ID NO:17 the FcγR3A engulfment signaling domain is truncated 1, 2, 3, 4, 5, or more amino acids at its N-terminus corresponding to the amino acid sequence of SEQ ID NO:18; the BAFF-R engulfment signaling domain is truncated 1, 2, 3, 4, 5, or more amino acids at its N-terminus corresponding to the amino acid sequence of SEQ ID NO:19; the DAP-12 engulfment signaling domain is truncated 1, 2, 3, 4, 5, or more amino acids at its N-terminus corresponding to the amino acid sequence of SEQ ID NO:20; the NFAM1 engulfment signaling domain is truncated 1, 2, 3, 4, 5, or more amino acids at its N-terminus corresponding to the amino acid sequence of SEQ ID NO:21; the CD79b engulfment signaling domain is truncated 1, 2, 3, 4, 5, or more amino acids at its N-terminus corresponding to the amino acid sequence of SEQ ID NO:84; the TLR1 engulfment signaling domain is truncated 1, 2, 3, 4, 5, or more amino acids at its N-terminus corresponding to the amino acid sequence of SEQ ID NO:23; the TLR2 engulfment signaling domain is truncated 1, 2, 3, 4, 5, or more amino acids at its N-terminus corresponding to the amino acid sequence of SEQ ID NO:24; the TLR3 engulfment signaling domain is truncated 1, 2, 3, 4, 5, or more amino acids at its N-terminus corresponding to the amino acid sequence of SEQ ID NO:25; the TLR4 engulfment signaling domain is truncated 1, 2, 3, 4, 5, or more amino acids at its N-terminus corresponding to the amino acid sequence of SEQ ID NO:26; the TLR5 engulfment signaling domain is truncated 1, 2, 3, 4, 5, or more amino acids at its N-terminus corresponding to the amino acid sequence of SEQ ID NO:27; the TLR6 engulfment signaling domain is truncated 1, 2, 3, 4, 5, or more amino acids at its N-terminus corresponding to the amino acid sequence of SEQ ID NO:28; the TLR7 engulfment signaling domain is truncated 1, 2, 3, 4, 5, or more amino acids at its N-terminus corresponding to the amino acid sequence of SEQ ID NO:29; the TLR8 engulfment signaling domain is truncated 1, 2, 3, 4, 5, or more amino acids at its N-terminus corresponding to the amino acid sequence of SEQ ID NO:30; the TLR9 engulfment signaling domain is truncated 1, 2, 3, 4, 5, or more amino acids at its N-terminus corresponding to the amino acid sequence of SEQ ID NO:31; the Traf2 engulfment signaling domain is truncated 1, 2, 3, 4, 5, or more amino acids at its N-terminus corresponding to the amino acid sequence of SEQ ID NO:32; or the Traf3 engulfment signaling domain is truncated 1, 2, 3, 4, 5, or more amino acids at its N-terminus corresponding to the amino acid sequence of SEQ ID NO:33.

In certain embodiments, the MRC1 engulfment signaling domain is truncated 1, 2, 3, 4, 5, or more amino acids at its C-terminus corresponding to the amino acid sequence of SEQ ID NO:5; the MERTK engulfment signaling domain is truncated 1, 2, 3, 4, 5, or more amino acids at its C-terminus corresponding to the amino acid sequence of SEQ ID NO:6; the Tyro3 engulfment signaling domain is truncated 1, 2, 3, 4, 5, or more amino acids at its C-terminus corresponding to the amino acid sequence of SEQ ID NO:7; the Axl engulfment signaling domain is truncated 1, 2, 3, 4, 5, or more amino acids at its C-terminus corresponding to the amino acid sequence of SEQ ID NO:8; the ELMO engulfment signaling domain is truncated 1, 2, 3, 4, 5, or more amino acids at its C-terminus corresponding to the amino acid sequence of SEQ ID NO:9; the Traf6 engulfment signaling domain is truncated 1, 2, 3, 4, 5, or more amino acids at its C-terminus corresponding to the amino acid sequence of SEQ ID NO:10; the Syk engulfment signaling domain is truncated 1, 2, 3, 4, 5, or more amino acids at its C-terminus corresponding to the amino acid sequence of SEQ ID NO:11; the MyD88 engulfment signaling domain is truncated 1, 2, 3, 4, 5, or more amino acids at its C-terminus corresponding to the amino acid sequence of SEQ ID NO:12; the FcϵRIγ engulfment signaling domain is truncated 1, 2, 3, 4, 5, or more amino acids at its C-terminus corresponding to the amino acid sequence of SEQ ID NO:14; the FcγR1 engulfment signaling domain is truncated 1, 2, 3, 4, 5, or more amino acids at its C-terminus corresponding to the amino acid sequence of SEQ ID NO:15 the FcγR2A engulfment signaling domain is truncated 1, 2, 3, 4, 5, or more amino acids at its C-terminus corresponding to the amino acid sequence of SEQ ID NO:16; the FcγR2C engulfment signaling domain is truncated 1, 2, 3, 4, 5, or more amino acids at its C-terminus corresponding to the amino acid sequence of SEQ ID NO:17; the FcγR3A engulfment signaling domain is truncated 1, 2, 3, 4, 5, or more amino acids at its C-terminus corresponding to the amino acid sequence of SEQ ID NO:18; the BAFF-R engulfment signaling domain is truncated 1, 2, 3, 4, 5, or more amino acids at its C-terminus corresponding to the amino acid sequence of SEQ ID NO:19; the DAP-12 engulfment signaling domain is truncated 1, 2, 3, 4, 5, or more amino acids at its C-terminus corresponding to the amino acid sequence of SEQ ID NO:20; the NFAM1 engulfment signaling domain is truncated 1, 2, 3, 4, 5, or more amino acids at its C-terminus corresponding to the amino acid sequence of SEQ ID NO:21; the CD79b engulfment signaling domain is truncated 1, 2, 3, 4, 5, or more amino acids at its C-terminus corresponding to the amino acid sequence of SEQ ID NO:84; the TLR1 engulfment signaling domain is truncated 1, 2, 3, 4, 5, or more amino acids at its C-terminus corresponding to the amino acid sequence of SEQ ID NO:23; the TLR2 engulfment signaling domain is truncated 1, 2, 3, 4, 5, or more amino acids at its C-terminus corresponding to the amino acid sequence of SEQ ID NO:24; the TLR3 engulfment signaling domain is truncated 1, 2, 3, 4, 5, or more amino acids at its C-terminus corresponding to the amino acid sequence of SEQ ID NO:25; the TLR4 engulfment signaling domain is truncated 1, 2, 3, 4, 5, or more amino acids at its C-terminus corresponding to the amino acid sequence of SEQ ID NO:26; the TLR5 engulfment signaling domain is truncated 1, 2, 3, 4, 5, or more amino acids at its C-terminus corresponding to the amino acid sequence of SEQ ID NO:27; the TLR6 engulfment signaling domain is truncated 1, 2, 3, 4, 5, or more amino acids at its C-terminus corresponding to the amino acid sequence of SEQ ID NO:28; the TLR7 engulfment signaling domain is truncated 1, 2, 3, 4, 5, or more amino acids at its C-terminus corresponding to the amino acid sequence of SEQ ID NO:29; the TLR8 engulfment signaling domain is truncated 1, 2, 3, 4, 5, or more amino acids at its C-terminus corresponding to the amino acid sequence of SEQ ID NO:30; the TLR9 engulfment signaling domain is truncated 1, 2, 3, 4, 5, or more amino acids at its C-terminus corresponding to the amino acid sequence of SEQ ID NO:31; the Traf2 engulfment signaling domain is truncated 1, 2, 3, 4, 5, or more amino acids at its C-terminus corresponding to the amino acid sequence of SEQ ID NO:32; or the Traf3 engulfment signaling domain is truncated 1, 2, 3, 4, 5, or more amino acids at its C-terminus corresponding to the amino acid sequence of SEQ ID NO:33.

In certain embodiments, a truncated MyD88 engulfment signaling domain comprises a death domain but lacks a Toll/interleukin-1 receptor (TIR) homology domain. An example of such a truncated MyD88 engulfment signaling domain comprises an amino acid sequence of SEQ ID NO:34. In certain embodiments, a truncated MyD88 engulfment signaling domain comprises a TIR domain. An example of a truncated MyD88 engulfment signaling domain comprising a TIR domain comprises an amino acid sequence of SEQ ID NO:86. An exemplary truncated Traf6 signaling domain comprises an amino acid sequence of SEQ ID NO:35. An exemplary truncated NFAM1 signaling domain comprises an amino acid sequence of SEQ ID NO:36. An exemplary truncated CD79b signaling domain comprises an amino acid sequence of SEQ ID NO:22.

In certain embodiments, a CER comprises a primary homeostatic engulfment signaling domain and a secondary engulfment signaling domain that are from the same molecule. In other embodiments, the primary homeostatic engulfment signaling domain and the secondary engulfment signaling domain are from different molecules.

In certain embodiments, signaling by the engulfment signaling domain (e.g., the homeostatic engulfment signaling domain or combination of a primary homeostatic engulfment signaling domain and secondary engulfment signaling domain) results in expression of at least one of an anti-inflammatory cytokine and immunosuppressive cytokine. Exemplary anti-inflammatory and immunosuppressive cytokines include TGF-β and IL-10.

Engulfment signaling domains may be derived from a mammalian species, including humans, primates, cows, horses, goats, sheep, dogs, cats, mice, rats, rabbits, guinea pigs, pigs, and transgenic species thereof.

### Transmembrane Domain

CERs of the present disclosure comprise a transmembrane domain that connects and is positioned between the extracellular domain and the engulfment signaling domain. The transmembrane domain is a hydrophobic alpha helix that transverses the host cell membrane and anchors the CER in the host cell membrane.

The transmembrane domain may be directly fused to the binding domain or to the extracellular spacer domain if present. In certain embodiments, the transmembrane domain is derived from an integral membrane protein (e.g., receptor, cluster of differentiation (CD) molecule, enzyme, transporter, cell adhesion molecule, or the like). The transmembrane domain can be selected from the same molecule as the extracellular domain or the engulfment signaling domain (e.g., a CER comprises a TLR4 engulfment signaling domain and a TLR4 transmembrane domain). In certain embodiments, the transmembrane domain and the extracellular domain are each selected from different molecules. In other embodiments, the transmembrane domain and the engulfment signaling domain are each selected from different molecules. In yet other embodiments, the transmembrane domain, the extracellular domain, and the engulfment signaling domain are each selected from different molecules.

In certain embodiments, the transmembrane domain comprises a Tim1, Tim4, Tim3, FcR (e.g., FcγR1, FcγR2A, FcγR2B2, FcγR2C, FcγR3A, Fcϵ1, or FcαR1), CD8a, CD28, MERTK, Axl, Tyro3, CD4, DAP12, MRC1, TLR1, TLR2, TLR3, TLR4, TLR5, TLR6, TLR7, TLR8, or TLR9 transmembrane domain.

In certain embodiments, the transmembrane domain comprises a sequence that has at least about 75%, 80%, 85%, 90%, 91%, 92%, 93%, 94%, 95%, 96%, 97%, 98%, 99%, 99.5%, or 100% identity to a Tim1 transmembrane domain comprising an amino acid sequence of SEQ ID NO:37, Tim4 transmembrane domain comprising an amino acid sequence of SEQ ID NO:38 or 81, Tim3 transmembrane domain comprising an amino acid sequence of SEQ ID NO:39, FcγR1 transmembrane domain comprising an amino acid sequence of SEQ ID NO:40, FcγR2A transmembrane domain comprising an amino acid sequence of SEQ ID NO:41, FcγR2B2 transmembrane domain comprising an amino acid sequence of SEQ ID NO:42, FcγR2C transmembrane domain comprising an amino acid sequence of SEQ ID NO:43, FcγR3A transmembrane domain comprising an amino acid sequence of SEQ ID NO:44, FcϵR1 transmembrane domain comprising an amino acid sequence of SEQ ID NO:45, FcαR1 transmembrane domain comprising an amino acid sequence of SEQ ID NO:46, CD8a transmembrane domain comprising an amino acid sequence of SEQ ID NO:47, CD28 transmembrane domain comprising an amino acid sequence of SEQ ID NO:48, MERTK transmembrane domain comprising an amino acid sequence of SEQ ID NO:49, Axl transmembrane domain comprising an amino acid sequence of SEQ ID NO:50, Tyro3 transmembrane domain comprising an amino acid sequence of SEQ ID NO:51, CD4 transmembrane domain comprising an amino acid sequence of SEQ ID NO:52, DAP12 transmembrane domain comprising an amino acid sequence of SEQ ID NO:53, MRC1 transmembrane domain comprising an amino acid sequence of SEQ ID NO:54, TLR1 transmembrane domain comprising an amino acid sequence of SEQ ID NO:55, TLR2 transmembrane domain comprising an amino acid sequence of SEQ ID NO:56, TLR3 transmembrane domain comprising an amino acid sequence of SEQ ID NO:57, TLR4 transmembrane domain comprising an amino acid sequence of SEQ ID NO:58, TLR5 transmembrane domain comprising an amino acid sequence of SEQ ID NO:59, TLR6 transmembrane domain comprising an amino acid sequence of SEQ ID NO:60, TLR7 transmembrane domain comprising an amino acid sequence of SEQ ID NO:61, TLR8 transmembrane domain comprising an amino acid sequence of SEQ ID NO:62, or TLR9 transmembrane domain comprising an amino acid sequence of SEQ ID NO:63.

In certain embodiments, the transmembrane domain is a Tim1 transmembrane domain comprising or consisting of an amino acid sequence of SEQ ID NO:37, Tim4 transmembrane domain comprising or consisting of an amino acid sequence of SEQ ID NO:38, Tim3 transmembrane domain comprising or consisting of an amino acid sequence of SEQ ID NO:39, FcγR1 transmembrane domain comprising or consisting of an amino acid sequence of SEQ ID NO:40, FcγR2A transmembrane domain comprising or consisting of an amino acid sequence of SEQ ID NO:41, FcγR2B2 transmembrane domain comprising or consisting of an amino acid sequence of SEQ ID NO:42, FcγR2C transmembrane domain comprising or consisting of an amino acid sequence of SEQ ID NO:43, FcγR3A transmembrane domain comprising or consisting of an amino acid sequence of SEQ ID NO:44, FcϵR1 transmembrane domain comprising or consisting of an amino acid sequence of SEQ ID NO:45, FcαR1 transmembrane domain comprising or consisting of an amino acid sequence of SEQ ID NO:46, CD8a transmembrane domain comprising or consisting of an amino acid sequence of SEQ ID NO:47, CD28 transmembrane domain comprising or consisting of an amino acid sequence of SEQ ID NO:48, MERTK transmembrane domain comprising or consisting of an amino acid sequence of SEQ ID NO:49, Axl transmembrane domain comprising or consisting of an amino acid sequence of SEQ ID NO:50, Tyro3 transmembrane domain comprising or consisting of an amino acid sequence of SEQ ID NO:51, CD4 transmembrane domain comprising or consisting of an amino acid sequence of SEQ ID NO:52, DAP12 transmembrane domain comprising or consisting of an amino acid sequence of SEQ ID NO:53, MRC1 transmembrane domain comprising or consisting of an amino acid sequence of SEQ ID NO:54, TLR1 transmembrane domain comprising or consisting of an amino acid sequence of SEQ ID NO:55, TLR2 transmembrane domain comprising or consisting of an amino acid sequence of SEQ ID NO:56, TLR3 transmembrane domain comprising or consisting of an amino acid sequence of SEQ ID NO:57, TLR4 transmembrane domain comprising or consisting of an amino acid sequence of SEQ ID NO:58, TLR5 transmembrane domain comprising or consisting of an amino acid sequence of SEQ ID NO:59, TLR6 transmembrane domain comprising or consisting of an amino acid sequence of SEQ ID NO:60, TLR7 transmembrane domain comprising or consisting of an amino acid sequence of SEQ ID NO:61, TLR8 transmembrane domain comprising or consisting of an amino acid sequence of SEQ ID NO:62, or TLR9 transmembrane domain comprising or consisting of an amino acid sequence of SEQ ID NO:63.

It is understood that direct fusion of one domain to another domain of a CER described herein does not preclude the presence of intervening junction amino acids. Junction amino acids may be natural or non-natural (e.g., resulting from the construct design of a chimeric protein).

In certain embodiments, a chimeric engulfment receptor comprises polynucleotide sequences derived from any mammalian species, including humans, primates, cows, horses, goats, sheep, dogs, cats, mice, rats, rabbits, guinea pigs, pigs, transgenic species thereof, or any combination thereof. In certain embodiments, a chimeric engulfment receptor is murine, human, chimeric (with sequences from two or more species) or humanized.

### Examples of CERs

The component parts of a CER as disclosed herein can be selected and arranged in various combinations to provide a desired specificity and engulfment phenotype to a host cell.

In certain embodiments, a CER of the present disclosure comprises an extracellular domain comprising a binding domain that binds to amyloid-β; an engulfment signaling domain comprising a homeostatic engulfment signaling domain; and a transmembrane domain positioned between and connecting the extracellular domain and the engulfment signaling domain. In certain embodiments, the extracellular domain further comprises an extracellular spacer domain, such as an IgG4 hinge region, positioned between the binding domain and the transmembrane domain. In further embodiments, the binding domain comprises a scFv derived from BIIB037 antibody, bapineuzumab, crenezumab, solanezumab, ponezumab, gantenerumab, or BAN-2401 antibody. In yet further embodiments, the transmembrane domain comprises a Tim4 transmembrane domain. In still further embodiments, the homeostatic engulfment signaling domain comprises a MERTK or Axl signaling domain.

An exemplary CER of the present disclosure comprises an extracellular domain comprising a binding domain comprising a β-amyloid specific scFv and an extracellular spacer domain comprising an IgG4 hinge region; an engulfment signaling domain comprising a MERTK signaling domain; a transmembrane domain comprising a Tim4 transmembrane domain positioned between and connecting the extracellular domain and the engulfment signaling domain; wherein the extracellular spacer domain is positioned between the binding domain and the transmembrane domain. In certain embodiments, such an exemplary CER comprises an amino acid sequence of SEQ ID NO:65.

Another exemplary CER of the present disclosure comprises an extracellular domain comprising a binding domain comprising a β-amyloid specific scFv and an extracellular spacer domain comprising an IgG4 hinge region; an engulfment signaling domain comprising a Axl signaling domain; a transmembrane domain comprising a Tim4 transmembrane domain positioned between and connecting the extracellular domain and the engulfment signaling domain; wherein the extracellular spacer domain is positioned between the binding domain and the transmembrane domain. In certain embodiments, such an exemplary CER comprises an amino acid sequence of SEQ ID NO:66.

In certain embodiments, a CER of the present disclosure comprises an extracellular domain comprising a binding domain that binds to Tau; an engulfment signaling domain comprising a homeostatic engulfment signaling domain; and a transmembrane domain positioned between and connecting the extracellular domain and the engulfment signaling domain. In certain embodiments, the extracellular domain further comprises an extracellular spacer domain, such as an IgG4 hinge region, positioned between the binding domain and the transmembrane domain. In further embodiments, the binding domain comprises a scFv derived from ABBV-8E12 antibody, BMS-986168 antibody, BIIB076 antibody, R07105705 antibody, or RG7345 antibody. In yet further embodiments, the transmembrane domain comprises a Tim4 transmembrane domain. In still further embodiments, the homeostatic engulfment signaling domain comprises a MERTK or Axl signaling domain.

In certain embodiments, a CER of the present disclosure comprises an extracellular domain comprising a binding domain that binds to a-synuclein; an engulfment signaling domain comprising a homeostatic engulfment signaling domain; and a transmembrane domain positioned between and connecting the extracellular domain and the engulfment signaling domain. In certain embodiments, the extracellular domain further comprises an extracellular spacer domain, such as an IgG4 hinge region, positioned between the binding domain and the transmembrane domain. In further embodiments, the binding domain comprises a BIIB054 scFv or 12F4 scFv. In yet further embodiments, the transmembrane domain comprises a Tim4 transmembrane domain. In still further embodiments, the homeostatic engulfment signaling domain comprises a MERTK or Axl signaling domain.

In certain embodiments, a CER of the present disclosure comprises an extracellular domain comprising a binding domain that binds to semaphorin 4D; an engulfment signaling domain comprising a homeostatic engulfment signaling domain; and a transmembrane domain positioned between and connecting the extracellular domain and the engulfment signaling domain. In certain embodiments, the extracellular domain further comprises an extracellular spacer domain, such as an IgG4 hinge region, positioned between the binding domain and the transmembrane domain. In further embodiments, the binding domain comprises a VX15 scFv. In yet further embodiments, the transmembrane domain comprises a Tim4 transmembrane domain. In still further embodiments, the homeostatic engulfment signaling domain comprises a MERTK or Axl signaling domain.

### Polynucleotides, Vectors, and Host Cells

In certain aspects, the present disclosure provides nucleic acid molecules that encode any one or more of the CERs described herein. A nucleic acid may refer to a single- or double-stranded DNA, cDNA, or RNA, and may include a positive and a negative strand of the nucleic acid which complement one another, including antisense

DNA, cDNA, and RNA. A nucleic acid may be naturally occurring or synthetic forms of DNA or RNA. The nucleic acid sequences encoding a desired CER can be obtained or produced using recombinant methods known in the art using standard techniques, such as by screening libraries from cells expressing the desired sequence or a portion thereof, by deriving the sequence from a vector known to include the same, or by isolating the sequence or a portion thereof directly from cells or tissues containing the same as described in, for example, Sambrook et al. (1989 and 2001 editions; Molecular Cloning: A Laboratory Manual, Cold Spring Harbor Laboratory Press, NY) and Ausubel et al. (Current Protocols in Molecular Biology, 2003). Alternatively, the sequence of interest can be produced synthetically, rather than being cloned.

Polynucleotides encoding the CER compositions provided herein may be derived from any animal, such as humans, primates, cows, horses, sheep, dogs, cats, mice, rats, rabbits, guinea pigs, pigs, or a combination thereof. In certain embodiments, a polynucleotide encoding the CER is from the same animal species as the host cell into which the polynucleotide is inserted.

The polynucleotides encoding CERs of the present disclosure may be operatively linked to expression control sequences. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. In certain embodiments, a polynucleotide encoding a CER comprises a sequence encoding a signal peptide (also referred to as leader peptide or signal sequence) at the 5′-end for targeting of the precursor protein to the secretory pathway. The signal peptide is optionally cleaved from the N-terminus of the extracellular domain during cellular processing and localization of the CER to the host cell membrane. A polypeptide from which a signal peptide sequence has been cleaved or removed may also be called a mature polypeptide. Examples of signal peptides that may be used in the CERs of the present disclosure include signal peptides derived from endogenous secreted proteins, including, e.g., GM-CSF (amino acid sequence of SEQ ID NO:68), Tim4 (amino acid sequence of SEQ ID NO:69). In certain embodiments, a polynucleotide sequence encodes a mature CER polypeptide, or a polypeptide sequence comprises a mature CER polypeptide. It is understood by persons of skill in the art that for sequences disclosed herein that include a signal peptide sequence, the signal peptide sequence may be replaced with another signal peptide that is capable of trafficking the encoded protein to the extracellular membrane.

In certain embodiments, a CER encoding polynucleotide of the present disclosure is codon optimized for efficient expression in a target host cell comprising the polynucleotide (see, e.g., Scholten et al., Clin. Immunol. 119:135-145 (2006)). As used herein, a “codon optimized” polynucleotide comprises a heterologous polynucleotide having codons modified with silent mutations corresponding to the abundances of tRNA in a host cell of interest.

A single polynucleotide molecule may encode one, two, or more CERs according to any of the embodiments disclosed herein. A polynucleotide encoding more than one transcript may comprise a sequence (e.g., IRES, viral 2A peptide) disposed between each transcript for multicistronic expression.

A polynucleotide encoding a desired CER can be inserted into an appropriate vector, e.g., a viral vector, non-viral plasmid vector, and non-viral vectors, such as lipid-based DNA vectors, modified mRNA (modRNA), self-amplifying mRNA, CELiD, and transposon-mediated gene transfer (PiggyBac, Sleeping Beauty), for introduction into a host cell of interest (e.g., an immune cell). Polynucleotides encoding a CER of the present disclosure can be cloned into any suitable vector, such as an expression vector, a replication vector, a probe generation vector, or a sequencing vector. In certain embodiments, a polynucleotide encoding the extracellular domain, a polynucleotide encoding the transmembrane domain, and a polynucleotide encoding the engulfment signaling domain are joined together into a single polynucleotide and then inserted into a vector. In other embodiments, a polynucleotide encoding the extracellular domain, a polynucleotide encoding the transmembrane domain, and a polynucleotide encoding the engulfment signaling domain may be inserted separately into a vector such that the expressed amino acid sequence produces a functional CER. A vector that encodes a CER is referred to herein as a “CER vector.”

In certain embodiments, a vector comprises a polynucleotide encoding one CER. In certain embodiments, a vector comprises one polynucleotide encoding two or more CERs. In certain embodiments, a single polynucleotide encoding two or more CERs is cloned into a cloning site and expressed from a single promoter, with each CER sequence separated from each other by an internal ribosomal entry site (IRES) or peptide cleavage site, such as a furin cleavage site or viral 2A self-cleaving peptide, to allow for co-expression of multiple proteins from a single open reading frame (e.g., a multicistronic vector). In certain embodiments, a viral 2A peptide is a porcine teschovirus-1 (P2A), Thosea asigna virus (T2A), equine rhinitis A virus (E2A), foot-and-mouth disease virus (F2A), or variant thereof. An exemplary T2A peptide comprises an amino acid sequence of any one of SEQ ID NOs:70 and 87-89. An exemplary P2A peptide comprises an amino acid sequence of SEQ ID NO:71 or 90. An exemplary E2A peptide sequence comprises an amino acid sequence of SEQ ID NO:72. An exemplary F2A peptide sequence comprises an amino acid sequence of SEQ ID NO:73.

In certain embodiments, a vector comprises two or more polynucleotides, each polynucleotide encoding a CER. The two or more polynucleotides encoding CERs may be cloned sequentially into a vector at different cloning sites, with each CER expressed under the regulation of different promoters. In certain embodiments, vectors that allow long-term integration of a transgene and propagation to daughter cells are utilized. Examples include viral vectors such as, adenovirus, adeno-associated virus, vaccinia virus, herpes viruses, cytomegalovirus, pox virus, or retroviral vectors, such as lentiviral vectors. Vectors derived from lentivirus can be used to achieve long-term gene transfer and have added advantages over vectors including the ability to transduce non-proliferating cells, such as hepatocytes, and low immunogenicity.

A vector that encodes a core virus is referred to herein as a “viral vector.” There are a large number of available viral vectors suitable for use with the compositions of the instant disclosure, including those identified for human gene therapy applications (see Pfeifer and Verma, Ann. Rev. Genomics Hum. Genet. 2:177, 2001). Suitable viral vectors include vectors based on RNA viruses, such as retrovirus-derived vectors, e.g., Moloney murine leukemia virus (MLV)-derived vectors, and include more complex retrovirus-derived vectors, e.g., lentivirus-derived vectors. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing chimeric receptor transgenes are known in the art and have been previous described, for example, in U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; Verhoeyen et al., Methods Mol. Biol. 506:97, 2009. Retroviral and lentiviral vector constructs and expression systems are also commercially available.

In certain embodiments, a viral vector is used to introduce a non-endogenous polynucleotide encoding a CER to a host cell. A viral vector may be a retroviral vector or a lentiviral vector. A viral vector may also include a nucleic acid sequence encoding a marker for transduction. Transduction markers for viral vectors are known in the art and include selection markers, which may confer drug resistance, or detectable markers, such as fluorescent markers or cell surface proteins that can be detected by methods such as flow cytometry. In particular embodiments, a viral vector further comprises a gene marker for transduction comprising a fluorescent protein (e.g., green, yellow), an extracellular domain of human CD2, or a truncated human EGFR (EGFRt or tEGFR; see Wang et al., Blood 118:1255, 2011). An exemplary tEGFR comprises an amino acid sequence of SEQ ID NO:74. When a viral vector genome comprises a plurality of genes to be expressed in a host cell as separate proteins from a single transcript, the viral vector may also comprise additional sequences between the two (or more) genes allowing for multicistronic expression. Examples of such sequences used in viral vectors include internal ribosome entry sites (IRES), furin cleavage sites, viral 2A peptides (e.g., T2A, P2A, E2A, F2A), or any combination thereof.

Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5: 1517, 1998).

Other viral vectors recently developed for gene therapy uses can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

In certain embodiments, a CER vector can be constructed to optimize spatial and temporal control. For example, CER vector can include promoter elements to optimize spatial and temporal control. In some embodiments, a CER vector includes tissue specific promoters or enhancers that enable specific induction of a CER to an organ (e.g., brain), a cell type (e.g., immune cell or microglial cell), or a pathologic microenvironment, such as amyloid plaques. An “enhancer” is an additional promoter element that can function either cooperatively or independently to activate transcription. In certain embodiments, a CER vector includes a constitutive promoter. In certain embodiments, a CER vector includes an inducible promoter. In certain embodiments, a

CER vector includes a tissue specific promoter.

Where temporal control is desired, a CER vector may include an element that allows for inducible depletion of transduced cells. For example, such a vector may include an inducible suicide gene. A suicide gene may be an apoptotic gene or a gene that confers sensitivity to an agent (e.g., a drug). Exemplary suicide genes include chemically inducible caspase 9 (iCASP9) (U.S. Patent Publication No. 2013/0071414), chemically inducible Fas, or Herpes simplex virus thymidine kinase (HSV-TK), which confers sensitivity to ganciclovir. In further embodiments, a CER vector can be designed to express a known cell surface antigen that, upon infusion of an associated antibody, enables depletion of transduced cells. Examples of cell surface antigens and their associated antibodies that may be used for depletion of transduced cells include CD20 and Rituximab, RQR8 (combined CD34 and CD20 epitopes, allowing CD34 selection and anti-CD20 deletion) and Rituximab, and EGFR and Cetuximab.

Inducible vector systems, such as the tetracycline (Tet)-On vector system which activates transgene expression with doxycycline (Heinz et al., Hum. Gene Ther. 2011, 22:166-76) may also be used for inducible CER expression. Inducible CER expression may be also accomplished via retention using a selective hook (RUSH) system based on streptavidin anchored to the membrane of the endoplasmic reticulum through a hook and a streptavidin binding protein introduced into the CER structure, where addition of biotin to the system leads to the release of the CER from the endoplasmic reticulum (Agaugue et al., 2015, Mol. Ther. 23(Suppl. 1):588).

In certain embodiments, a CER modified host cell may also be modified to co-express one or more small GTPases. Rho GTPases, a family of small (-21 k Da) signaling G proteins and also a subfamily of the Ras superfamily, regulate actin cytoskeleton organization in various cell types and promote pseudopod extension and phagosome closure during phagocytosis (see, e.g., Castellano et al., 2000, J. Cell Sci. 113:2955-2961). Engulfment requires F-actin recruitment beneath tethered cells or particles, and F-actin rearrangement to allow membrane extension resulting in cell or particle internalization. RhoGTPases include RhoA, Rac1, Rac2, RhoG, and CDC42. Other small GTPases, such as Rap1, is involved in regulation of complement mediated phagocytosis. Co-expression of a small GTPase with the CER may promote or enhance target cell or particle internalization and/or phagosome formation by the host cell. In some embodiments, a recombinant nucleic acid molecule encoding a GTPase is encoded on a separate vector than the CER-containing vector. In other embodiments, a recombinant nucleic acid molecule encoding a GTPase is encoded on the same vector as the CER. The GTPase and CER may be expressed under the regulation of different promoters on the same vector (e.g., at different multiple cloning sites). Alternatively, the CER and GTPase may be expressed under the regulation of one promoter in a multicistronic vector. The polynucleotide sequence encoding the CER and the polynucleotide sequence encoding the small GTPase(s) may be separated from each other by an IRES or viral 2A peptide in a multicistronic vector. Exemplary 2A peptides include T2A (SEQ ID NOS:70, 87, 88, and 90), P2A (SEQ ID NOS:71 and 90), E2A (SEQ ID NO:72), F2A (SEQ ID NO:73).

Examples of GTPases that may be co-expressed with a CER include Rac1, Rac2, Rab5 (also referred to as Rab5a), Rab7, Rap1, RhoA, RhoG, CDC42, or any combination thereof. In specific embodiments, the GTPase comprises or is a sequence that is at least 90%, at least 91%, at least 92%, at least 93%, at least 94%, at least 95%, at least 96%, at least 97%, at least 98%, at least 99%, at least 99.5%, or 100% identical to a Rac1 amino acid sequence of SEQ ID NO:75, a Rab5 amino acid sequence of SEQ II) NO:76, a Rab7 amino acid sequence of SEQ 113 NO:77, a Rap1 amino acid sequence of SEQ ID NO:78, a RhoA amino acid sequence of SEQ ID NO:79, a CDC42 amino acid sequence of SEQ ID NO:80, or any combination thereof.

In certain embodiments, a cell, such as an immune cell, obtained from a subject may be modified into a non-natural or recombinant cell (e.g., a non-natural or recombinant immune cell) by introducing a polynucleotide that encodes a CER as described herein and whereby the cell expresses a cell surface localized CER. In certain embodiments, a host cell is an immune cell, such as a myeloid progenitor cell or a lymphoid progenitor cell. Exemplary immune cells that may be modified to comprise a polynucleotide encoding a CER or a vector comprising a polynucleotide encoding a CER include a T cell, a natural killer cell, a B cell, a lymphoid precursor cell, an antigen presenting cell, a dendritic cell, a Langerhans cell, a myeloid precursor cell, a mature myeloid cell, a monocyte, a macrophage, or a microglial cell.

In certain embodiments, B cells are modified to express one or more CERs. B cells possess certain properties that may be advantageous as host cells, including: trafficking to sites of inflammation, capable of internalizing and presenting antigen, capable of costimulating T cells, highly proliferative, and self-renewing (persist for life). In certain embodiments, CER modified B cells are capable of digesting an engulfed target cell or engulfed target particle into smaller peptides and presenting them to T cells via an MHC molecule. Antigen presentation by CER modified B cells may contribute to antigen spreading of the immune response to non-targeted antigens. B cells include progenitor or precursor cells committed to the B cell lineage (e.g., pre-pro-B cells, pro-B cells, and pre-B cells); immature and inactivated B cells or mature and functional or activated B cells. In certain embodiments, B cells may be naive B cells, plasma cells, regulatory B cells, marginal zone B cells, follicular B cells, lymphoplasmacytoid cell, plasmablast cell, memory B cells, or any combination thereof. Memory B cells may be distinguished from naive B cells by expression of CD27, which is absent on naive B cells. In certain embodiments, the B cells can be primary cells or cell lines derived from human, mouse, rat, or other mammals. B cell lines are well known in the art. If obtained from a mammal, a B cell can be obtained from numerous sources, including blood, bone marrow, spleen, lymph node, or other tissues or fluids. A B cell composition may be enriched or purified.

In certain embodiments, T cells are modified to express one or more CERs. Exemplary T cells include CD4⁺ helper, CD8⁺ effector (cytotoxic), naïve (CD45 RA+, CCR7+, CD62L+, CD27+, CD45RO−), central memory (CD45RO⁺, CD62L⁺, CD8⁺), effector memory (CD45RA+, CD45RO−, CCR7−, CD62L−, CD27−), T memory stem, regulatory, mucosal-associated invariant (MAIT), γδ (gd), tissue resident T cells, natural killer T cells, or any combination thereof. In certain embodiments, the T cells can be primary cells or cell lines derived from human, mouse, rat, or other mammals. If obtained from a mammal, a T cell can be obtained from numerous sources, including blood, bone marrow, lymph node, thymus, or other tissues or fluids.

A T cell composition may be enriched or purified. T cell lines are well known in the art, some of which are described in Sandberg et al., Leukemia 21:230, 2000. In certain embodiments, the T cells lack endogenous expression of a TCRα gene, TCRβ gene, or both. Such T cells may naturally lack endogenous expression of TCRα and β chains or may have been modified to block expression (e.g., T cells from a transgenic mouse that does not express TCR α and β chains or cells that have been manipulated to inhibit expression of TCR α and β chains) or to knockout TCRα chain, TCRβ chain, or both genes.

In certain embodiments, host cells expressing a chimeric protein of this disclosure on the cell surface are not T cells or cells of a T cell lineage, but cells that are progenitor cells, stem cells or cells that have been modified to express cell surface anti-CD3.

In certain embodiments, microglial cells are modified to express one or more CERs. Microglia are located in the brain and spinal cord and are the main immune defense in the CNS. Microglial cells are capable of a variety of immune functions, including phagocytosis, antigen presentation to T cells, cytotoxicity, and cytokine secretion.

In certain embodiments, gene editing methods are used to modify the host cell genome to comprise a polynucleotide encoding a CER of the present disclosure. Gene editing, or genome editing, is a method of genetic engineering wherein DNA is inserted, replaced, or removed from a host cell's genome using genetically engineered endonucleases. The nucleases create specific double-stranded breaks at targeted loci in the genome. The host cell's endogenous DNA repair pathways then repair the induced break(s), e.g., by non-homologous ending joining (NHEJ) and homologous recombination. Exemplary endonucleases useful in gene editing include a zinc finger nuclease (ZFN), a transcription activator-like effector (TALE) nuclease, a clustered regularly interspaced short palindromic repeats (CRISPR)/Cas nuclease system (e.g., CRISPR-Cas9), a meganuclease, or combinations thereof. Methods of disrupting or knocking out genes or gene expression in immune cells including B cells and T cells, using gene editing endonucleases are known in the art and described, for example, in PCT Publication Nos. WO 2015/066262; WO 2013/074916; WO 2014/059173; Cheong et al., Nat. Comm. 2016 7:10934; Chu et al., Proc. Natl. Acad. Sci. USA 2016 113:12514-12519; methods from each of which are incorporated herein by reference in their entirety. In certain embodiments, expression of an endogenous gene of the host cell is inhibited, knocked down, or knocked out. Examples of endogenous genes that may be inhibited, knocked down, or knocked out in a B cell include IGH, IGκ, IGλ, or any combination thereof. Examples of endogenous genes that may be inhibited, knocked down, or knocked out in a T cell include a TCR gene (TRA or TRB), an HLA gene (HLA class I gene or HLA class II gene), an immune checkpoint molecule (PD-L1, PD-L2, CD80, CD86, B7-H3, B7-H4, HVEM, adenosine, GALS, VISTA, CEACAM-1, CEACAM-3, CEACAM-5, PVRL2, PD-1, CTLA-4, BTLA, KIR, LAG3, TIM3, A2aR, CD244/2B4, CD160, TIGIT, LAIR-1, or PVRIG/CD112R), or any combination thereof. Expression of an endogenous gene may be inhibited, knocked down, or knocked out at the gene level, transcriptional level, translational level, or a combination thereof. Methods of inhibiting, knocking down, or knocking out an endogenous gene may be accomplished, for example, by RNA interference agents (e.g., siRNA, shRNA, miRNA, etc.) or engineered endonucleases (e.g., CRISPR/Cas nuclease system, a zinc finger nuclease (ZFN), a Transcription Activator Like Effector nuclease (TALEN), a meganuclease), or any combination thereof. In certain embodiments, an endogenous B cell gene (e.g., IGH, IGκ, or IGλ) is knocked out by insertion of a polynucleotide encoding a CER of the present disclosure into the locus of the endogenous B cell gene, such as via an engineered endonuclease. In certain embodiments, an endogenous T cell gene (e.g., a TCR gene, an HLA gene, or an immune checkpoint molecule gene) is knocked out by insertion of a polynucleotide encoding a CER of the present disclosure into the locus of the endogenous T cell gene, such as via an engineered endonuclease.

In certain embodiments, a host cell may be modified to express one type of CER. In other embodiments, a host cell may express at least two or more different CERs.

The present disclosure also provides a composition comprising a population of CER modified host cells. In certain embodiments, the population of CER modified host cells may be a population of B cells, a population of T cells, a population of natural killer cells, a population of lymphoid precursor cells, a population of antigen presenting cells, a population of dendritic cells, a population of Langerhans cells, a population of myeloid precursor cells, a population of mature myeloid cells, a population of microglial cells, or any combination thereof. Furthermore, a population of CER modified host cells of a particular cell type may be composed of one or more subtypes. For example, a population of B cells may be composed of CER modified naïve B cells, plasma cells, regulatory B cells, marginal zone B cells, follicular B cells, lymphoplasmacytoid cell, plasmablast cell, memory B cells, or any combination thereof. In another example, a population of T cells may be composed of CER modified CD4⁺ helper T cells, CD8⁺ effector (cytotoxic) T cells, naïve (CD45 RA+, CCR7+, CD62L+, CD27+, CD45RO−) T cells, central memory (CD45RO⁺, CD62L⁺, CD8⁺) T cells, effector memory (CD45RA+, CD45RO−, CCR7−, CD62L−, CD27−) T cells, T memory stem cells, regulatory T cells, mucosal-associated invariant T cells (MAIT), γδ (gd), tissue resident T cells, natural killer T cells, or any combination thereof.

In certain embodiments, a population of host cells is composed of cells that express the same CER or set of CERs. In other embodiments, a population of host cells is composed of a mixture of two or more subpopulation of host cells, wherein each subpopulation expresses a different CER or set of CERs.

In certain embodiments, when preparing CER modified host cells, e.g., B cells or T cells, one or more growth factor cytokines that promotes proliferation of the host cells, e.g., B cells or T cells, may be added to the cell culture. The cytokines may be human or non-human. Exemplary growth factor cytokines that may be used to promote T cell proliferation include IL-2, IL-15, or the like. Exemplary growth factor cytokines that may be used to promote B cell proliferation include CD40L, IL-2, IL-4, IL-15, IL-21, BAFF, or the like. Prior to genetic modification of the host cells with a CER vector, a source of host cells (e.g., T cells, B cells, natural killer cells, etc.) is obtained from a subject (e.g., whole blood, peripheral blood mononuclear cells, bone marrow, lymph node tissue, cord blood, thymus tissue, tissue from a site of infection, ascites, pleural effusion, spleen tissue), from which host cells are isolated using methods known in the art. Specific host cell subsets can be collected in accordance with known techniques and enriched or depleted by known techniques, such as affinity binding to antibodies, flow cytometry and/or immunomagnetic selection. After enrichment and/or depletion steps and introduction of a CER, in vitro expansion of the desired modified host cells can be carried out in accordance with known techniques, or variations thereof that will be apparent those skilled in the art.

In certain embodiments, a CER modified host cell has a phagocytic index of about 20 to about 1,500 for a target cell, antigen, protein, peptide, or particle. A “phagocytic index” is a measure of phagocytic activity of the transduced host cell as determined by counting the number of target cells, antigens, proteins, peptides, or particles ingested per CER modified host cell during a set period of incubation of a suspension of target cells or particles and CER modified host cells in media. Phagocytic index may be calculated by multiplying [total number of engulfed target cells, antigens, proteins, peptides, or particles/total number of counted CER modified cells (e.g., phagocytic frequency)]×[average area of target cell, antigen, protein, peptide, or particle staining per CER⁺ host cell×100 (e.g., hybrid capture)] or [total number of engulfed cells, antigens, proteins, peptides, or particles/total number of counted CER modified host cells]×[number of CER modified host cells containing engulfed cells, antigens, proteins, peptides, or particles/ total number of counted CER cells] x 100. In certain embodiments, a CER modified cell has a phagocytic index of about 30 to about 1,500; about 40 to about 1,500; about 50 to about 1,500; about 75 to about 1,500; about 100 to about 1,500; about 200 to about 1,500; about 300 to about 1,500; about 400 to about 1,500; about 500 to about 1,500; about 20 to about 1,400; about 30 to about 1,400; about 40 to about 1,400; about 50 to about 1,400; about 100 to about 1,400; about 200 to about 1,400; about 300 to about 1,400; about 400 to about 1,400; about 500 to about 1,400; about 20 to about 1,300; about 30 to about 1,300;

about 40 to about 1,300; about 50 to about 1,300; about 100 to about 1,300; about 200 to about 1,300; about 300 to about 1,300; about 400 to about 1,300; about 500 to about 1,300; about 20 to about 1,200; about 30 to about 1,200; about 40 to about 1,200; about 50 to about 1,200; about 100 to about 1,200; about 200 to about 1,200; about 300 to about 1,200; about 400 to about 1,200; about 500 to about 1,200; about 20 to about 1,100; about 30 to about 1,100; about 40 to about 1,100; about 50 to about 1,100; about 100 to about 1,100; about 200 to about 1,100; about 300 to about 1,100; about 400 to about 1,100; or about 500 to about 1,100; about 20 to about 1,000; about 30 to about 1,000; about 40 to about 1,000; about 50 to about 1,000; about 100 to about 1,000; about 200 to about 1,000; about 300 to about 1,000; about 400 to about 1,000; or about 500 to about 1,000; about 20 to about 750; about 30 to about 750; about 40 to about 750; about 50 to about 750; about 100 to about 750; about 200 to about 750; about 300 to about 750; about 400 to about 750; or about 500 to about 750; about 20 to about 500; about 30 to about 500; about 40 to about 500; about 50 to about 500; about 100 to about 500; about 200 to about 500; or about 300 to about 500. In further embodiments, the incubation time is from about 2 hours to about 4 hours, e.g., about 2 hours, about 3 hours, or about 4 hours. In yet further embodiments, a CER modified cell exhibits phagocytic index that is statistically significantly higher than a control cell transduced with truncated EGFR. Phagocytic index may be calculated using methods known in the art and as further described in the Examples, including quantification by flow cytometry or fluorescence microscopy.

Host cells may be from an animal, such as a human, primate, cow, horse, sheep, dog, cat, mouse, rat, rabbit, guinea pig, pig, or a combination thereof. In a preferred embodiment, the animal is a human. Host cells may be obtained from a healthy subject or a subject having a disease associated with expression or overexpression of an antigen.

### Methods of Use

The present disclosure provides methods for altering the engulfment phenotype of a cell comprising introducing into a host cell a nucleic acid molecule encoding at least one CER or a vector comprising at least one CER according to any of the embodiments described herein; and expressing the at least one CER in the host cell, wherein the at least one CER confers an engulfment phenotype specific to a neurodegenerative disease antigen that is not naturally targeted by the host cell. In certain embodiments, the engulfment phenotype is phagocytosis, wherein the engulfed target cell, particle, prion, extracellular protein or peptide is degraded.

In another aspect, the present disclosure provides a population of cells comprising introducing into a population of host cells a nucleic acid molecule encoding at least one CER or a vector comprising at least one CER according to any of the embodiments described herein; and expressing the at least one CER in the population of host cells, wherein the at least one CER confers an engulfment phenotype specific to a neurodegenerative disease antigen that is not naturally targeted by the host cells. In certain embodiments, the engulfment phenotype is phagocytosis, wherein the engulfed target cell, particle, prion, extracellular protein or peptide is degraded.

In yet another aspect, the present disclosure provides methods for enhancing the engulfment phenotype of a cell comprising introducing into a host cell a nucleic acid molecule encoding at least one CER or a vector comprising at least one CER according to any of the embodiments described herein; and expressing the at least one CER in the host cell, wherein the at least one CER is specific to a neurodegenerative disease antigen that is naturally targeted by the host cell and expression of the at least one CER by the host cell enhances the engulfment by the cell of a target cell, prion, particle, extracellular protein or peptide expressing the neurodegenerative disease antigen. In certain embodiments, the extracellular target protein or peptide is in its native conformation, misfolded, oligomerized, fibrillized, or aggregated. In certain embodiments, the engulfment phenotype is enhanced at least about 10%, 15%, 20%, 25%, 30%, 35%, 40%, 50%, 55%, 60%, 65%, 70%, 75%, 80% , 85%, 90%, 95%, 100%, 110%, 120%, 130%, 140%, 150%, 160%, 170%, 180%, 190%, 200% or more as compared to host cell that is not modified with a nucleic acid molecule encoding a CER or a vector comprising a CER.

In yet another aspect, the present disclosure provides methods for enhancing the engulfment phenotype of a population of cells comprising introducing into a population of host cells a nucleic acid molecule encoding at least one CER or a vector comprising at least one CER according to any of the embodiments described herein; and expressing the at least one CER in the population of host cells, wherein the at least one CER is specific to a neurodegenerative disease antigen that is naturally targeted by the host cells and expression of the at least one CER by the host cells enhances the engulfment by the host cells of a target cell, prion, particle, extracellular protein or peptide expressing the neurodegenerative disease antigen. In certain embodiments, the engulfment phenotype is phagocytosis, wherein the engulfed target cell, particle, prion, particle, extracellular protein or peptide is degraded. In certain embodiments, the extracellular target protein or peptide is in its native conformation, misfolded, oligomerized, fibrillized, or aggregated. In certain embodiments, the engulfment phenotype is enhanced at least about 10%, 15%, 20%, 25%, 30%, 35%, 40%, 50%, 55%, 60%, 65%, 70%, 75%, 80% , 85%, 90%, 95%, 100%, 110%, 120%, 130%, 140%, 150%, 160%, 170%, 180%, 190%, 200% or more as compared to a population of host cell that is not modified with a nucleic acid molecule encoding a CER or a vector comprising a CER.

CERs, nucleic acid molecules encoding CERs, vectors comprising CERs, and host cells that express CERs of the present disclosure may also be used in a method treating a subject suffering from a neurodegenerative disease or disorder. Embodiments of these methods include administering to a subject a therapeutically effective amount of a pharmaceutical composition comprising a CER, CER encoding nucleic acid molecule, CER vector, or CER modified host cell according to the present description.

Neurodegenerative diseases or disorders that may be treated using the CER compositions of the present disclosure include Lewy body disease, postpoliomyelitis syndrome, Shy-Draeger syndrome, olivopontocerebellar atrophy, Parkinson's disease, multiple system atrophy, striatonigral degeneration, frontotemporal lobar degeneration with ubiquitinated inclusions (FLTD-U), tauopathies (including, but not limited to, Alzheimer disease and supranuclear palsy), prion diseases (also known as transmissible spongiform encephalopathies, including, but not limited to, bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease (including Amyotrophic lateral sclerosis (Lou Gherig's disease)), and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia). In certain embodiments, the CER compositions of the present disclosure provide methods for reducing or preventing aberrant protein accumulation or aggregation associated with a neurodegenerative disease. Many neurodegenerative diseases, including Alzheimer's disease, Parkinson's disease, Huntington's disease, Amyotrophic lateral sclerosis (Lou Gehrig's disease), and prion diseases, share a neuropathological signature, the aberrant accumulation or aggregation of proteins. For example, aggregation of amyloid-β or tau is involved in the pathogenesis of Alzheimer's disease. In another example, aggregation of Tau is involved in the pathogenesis of frontotemporal demention and other tauopathies. In another example, aggregation of a-synuclein is involved in the pathogenesis of Parkinson's disease (PD), dementia with Lewy bodies, multiple system atrophy, and Alzheimer's disease. In yet another example, aggregation of huntingtin is involved in the pathogenesis of Huntington's disease. In another example, SOD1, ataxin-2, or TDP-43 aggregation is involved in the pathogenesis of Amyotrophic lateral sclerosis. In another example, TDP-43 aggregation is involved in the pathogenesis of frontotemporal lobar degeneration (FLTD-U). In another example, aggregation of PrP^{Sc} is involved in the aggregation of prion diseases. Thus, in certain embodiments, CER therapy may be designed to target the disease-associated protein in order to reduce or prevent aberrant protein accumulation, thereby slowing or preventing progression of the neurodegenerative disease.

A CER of the present disclosure may be administered to a subject in cell-bound form (e.g., gene therapy of target cell population). Thus, for example, a CER of the present disclosure may be administered to a subject expressed on the surface of immune cells, e.g., T cells, Natural Killer Cells, Natural Killer T cells, B cells, lymphoid precursor cells, antigen presenting cells, dendritic cells, Langerhans cells, myeloid precursor cells, mature myeloid cells, microglial cells, including subsets thereof, or any combination thereof. In certain embodiments, methods of treating a subject comprise administering an effective amount of CER modified cells (i.e., recombinant cells that express one or more CERs). The CER modified cells may be xenogeneic, syngeneic, allogeneic, or autologous to the subject.

Pharmaceutical compositions including a CER modified cells may be administered in a manner appropriate to the disease or condition to be treated (or prevented) as determined by persons skilled in the medical art. An appropriate dose, suitable duration, and frequency of administration of the compositions will be determined by such factors as the condition of the patient, size, weight, body surface area, age, sex, type and severity of the disease, particular therapy to be administered, particular form of the active ingredient, time and the method of administration, and other drugs being administered concurrently. The present disclosure provides pharmaceutical compositions comprising CER modified cells and a pharmaceutically acceptable carrier, diluent, or excipient. Suitable excipients include water, saline, dextrose, glycerol, or the like and combinations thereof. Other suitable infusion medium can be any isotonic medium formulation, including saline, Normosol R (Abbott), Plasma-Lyte A (Baxter), 5% dextrose in water, or Ringer's lactate.

A treatment effective amount of cells in a pharmaceutical composition is at least one cell (for example, one CER modified B cell) or is more typically greater than 10² cells, for example, up to 10⁶, up to 10⁷, up to 10⁸ cells, up to 10⁹ cells, up to 10¹⁰ cells, or up to 10¹¹ cells or more. In certain embodiments, the cells are administered in a range from about 10⁶ to about 10¹⁰ cells/m², preferably in a range of about 10⁷ to about 10⁹ cells/m². The number of cells will depend upon the ultimate use for which the composition is intended as well the type of cells included therein. For example, a composition comprising cells modified to contain a CER specific for a particular neurodegenerative disease antigen will comprise a cell population containing from about 5% to about 95% or more of such cells. In certain embodiments, a composition comprising CER modified cells comprises a cell population comprising at least 5%, 10%, 15%, 20%, 25%, 30%, 35%, 40%, 45%, 50%, 55%, 60%, 65%, 70%, 75%, 80%, 85%, 90%, 95% or more of such cells. For uses provided herein, the cells are generally in a volume of a liter or less, 500 mls or less, 250 mls or less, or 100 mls or less. Hence the density of the desired cells is typically greater than 10⁴ cells/ml and generally is greater than 10⁷ cells/ml, generally 10⁸ cells/ml or greater. The cells may be administered as a single infusion or in multiple infusions over a range of time. Repeated infusions of CER modified cells may be separated by days, weeks, months, or even years if relapses of disease or disease activity are present. A clinically relevant number of immune cells can be apportioned into multiple infusions that cumulatively equal or exceed 10⁶, 10⁷, 10⁸, 10⁹, 10¹⁰, or 10¹¹ cells. A preferred dose for administration of a host cell comprising a recombinant expression vector as described herein is about 10⁷ cells/m², about 5×10⁷ cells/m², about 10⁸ cells/m², about 5×10⁸ cells/m², about 10⁹ cells/m², about 5×10⁹ cells/m², about 10¹⁰ cells/m², about 5×10¹⁰ cells/m², or about 10¹¹ cells/m². In certain embodiments, a composition of CER modified B cells and a composition of CER modified T cells are both administered, which administration may be simultaneous, concurrent or sequential.

In some embodiments, a composition as described herein is administered intravenously, intraperitoneally, intranasally, intrathecally, into the bone marrow, into the lymph node, into the brain, and/or into cerebrospinal fluid.

In certain embodiments, CERs of the present disclosure may be administered to a subject in combination with one or more additional therapeutic agents. Such additional therapeutic agents include an antibody, small molecule, peptide, aptamer, or protein. Examples of additional therapeutic agents include an NMDA receptor antagonist (e.g., memantine), monoamine depletor (e.g., tetrabenazine); an ergoloid mesylate; an anticholinergic antiparkinsonism agent (e.g., procyclidine, diphenhydramine, trihexylphenidyl, benztropine, biperiden and trihexyphenidyl); a dopaminergic antiparkinsonism agent (e.g., entacapone, selegiline, pramipexole, bromocriptine, rotigotine, selegiline, ropinirole, rasagiline, apomorphine, carbidopa, levodopa, pergolide, tolcapone and amantadine); a tetrabenazine; an anti-inflammatory agent (including, but not limited to, a nonsteroidal anti-inflammatory drug (e.g., indomethicin and other compounds listed above); a hormone (e.g., estrogen, progesterone and leuprolide); a vitamin (e.g., folate and nicotinamide); a dimebolin; a homotaurine (e.g., 3-aminopropanesulfonic acid; 3APS); a serotonin receptor activity modulator (e.g., xaliproden); an interferon; a glucocorticoid; corticosteroid; an amyloid-β aggregation inhibitor; BACE inhibitor; Tau inhibitor; protein misfolding inhibitor; atypical anti-psychotic drug; neuron- transmission enhancers; psychotherapeutic drugs; acetylcholine esterase inhibitors; calcium-channel blockers; biogenic amines; benzodiazepine tranquillizers; acetylcholine synthesis; storage or release enhancers; acetylcholine postsynaptic receptor agonists; monoamine oxidase-A or -B inhibitors; N-methyl-D-aspartate glutamate receptor antagonists; nonsteroidal anti-inflammatory drugs; antioxidants; cholinesterase inhibitors; or serotonergic receptor antagonists. Exemplary amyloid-β aggregation inhibitors include ELND-005 (also referred to as AZD-103 or scyllo-inositol), tramiprosate, and PTI-80 (Exebryl-1®; ProteoTech). Exemplary BACE inhibitors include E-2609 (Biogen, Eisai Co., Ltd.), AZD3293 (also known as LY3314814; AstraZeneca, Eli Lilly & Co.), MK-8931 (verubecestat), and JNJ-54861911 (Janssen, Shionogi Pharma). Exemplary Tau inhibitors include methylthioninium, LMTX (also known as leuco-methylthioninium or Trx- 0237; TauRx Therapeutics Ltd.), Rember™ (methylene blue or methylthioninium chloride [MTC]; Trx-0014; TauRx Therapeutics Ltd), PBT2 (Prana Biotechnology), and PTI-51-CH3 (TauPro™; ProteoTech). An exemplary protein misfolding inhibitor is NPT088 (euroPhage Pharmaceuticals). Exemplary atypical anti-psychotic drugs include clozapine, ziprasidone, risperidone, aripiprazole, and olanzapine.

In certain embodiments where CER modified cells are administered in combination with one or more additional therapies, the one or more additional therapies may be administered at a subtherapeutic dose due to an additive or synergistic effect of the combination with CER therapy. Combination therapy includes administration of a CER before an additional therapy (e.g., about 1-30 days before the additional therapy), concurrently with an additional therapy (on the same day), or after an additional therapy (e.g., about 1-30 days after the additional therapy). In certain embodiments, the CER modified cells are administered after administration of the one or more additional therapies. In further embodiments, the CER modified cells are administered 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days before or after administration of the one or more additional therapies. In still further embodiments, the CER modified cells are administered within 4 weeks, within 3 weeks, within 2 weeks, or within 1 week before or after administration of the one or more additional therapies. Where the one or more additional therapies involves multiple doses, the CER modified cells may be administered after the initial dose of the one or more additional therapies, after the final dose of the one or more additional therapies, or in between doses of the one or more additional therapies.

In certain embodiments, methods of the present disclosure include a depletion step. A depletion step to remove CERs from the subject may occur after a sufficient amount of time for therapeutic benefit in order to mitigate toxicity to a subject. In such embodiments, the CER vector includes an inducible suicide gene, such as iCASP9, inducible Fas, or HSV-TK, whose expression is switched on at the desired time to kill the CER modified host cell. Similarly, a CER vector may be designed for expression of a known cell surface antigen such as CD20 or truncated EGFR (SEQ ID NO:74) that facilitates depletion of transduced cells through infusion of an associated monoclonal antibody (mAb), for example, Rituximab for CD20 or Cetuximab for EGFR. Alemtuzumab, which targets CD52 present on the surface of mature lymphocytes, may also be used to deplete CER transduced B cells, T cells, or natural killer cells.

### EXAMPLES

### Example 1

### Construction of CER64 and CER65

An anti-amyloid-β single chain fragment variable (scFv) derived from

BIIB037 antibody (aducanumab) (SEQ ID NO:2) was fused to an IgG4 hinge region (SEQ ID NO:3), a Tim4 transmembrane domain (SEQ ID NO:81), and a MERTK signaling domain (SEQ ID NO:85) to create a chimeric engulfment receptor “CER64” (B1113037 scFv-IgG4-Tim4-MERTK) having an amino acid sequence of SEQ ID NO:65. The MERTK signaling domain transduces a signal for engulfment, and the

BIIB037 scFv is selected for binding aggregated forms of amyloid-β. The CER64 polynucleotide sequence was inserted into the pLenti lentiviral vector along with truncated EGFR (tEGFR) as a transduction marker, separated by a T2A sequence. Human primary B cells or mouse Ba/F3 B cells were transduced with pLenti vector expressing CER64 and tEGFR, expanded, sorted by FACs, and used for in vitro studies.

An anti-amyloid-β single chain fragment variable (scFv) derived from BIIB037 antibody (aducanumab) (SEQ ID NO:2) was fused to an IgG4 hinge region (SEQ ID NO:3), a Tim4 transmembrane domain (SEQ ID NO:81), and an Axl signaling domain (SEQ ID NO:8) to create a chimeric engulfment receptor “CER65” (BIIB037 scFv-IgG4-Tim4-Axl) having an amino acid sequence of SEQ ID NO:66. The Axl signaling domain transduces a signal for engulfment, and the BIIB037 scFv is selected for binding aggregated forms of amyloid-β.

Under normal conditions, the B cells lack the capacity to engulf target cells and were therefore selected to establish an assay system for engulfment. To transduce Ba/F3 cells or human primary B cells, 100 μl of viral vector expressing CER64 and 5 μl TRANSDUX™ transduction reagent were diluted in 0.5 ml Complete Cell Growth Media and added to the host cells. The host cells were then centrifuged at 270×g rpm for 1 hour in a 32° C. pre-warmed centrifuge. The host cells were incubated for 24 hours at 37° C. The cells were expanded for another 48 hours in appropriate cell culture media. Positive cell transductants were sorted using fluorescence activated cell sorting (FACs) (Sony Sorter SH800) by staining with a labeled EGFR-specific antibody (Cetixumab). Post sorting, purified, transduced cells comprising the CER64 containing viral vector were rested for 48 hours prior to being utilized for phagocytic assays.

### Example 2

### Emgulfment of Amyloid-B 42 Peptide by CER64 Modified B Cells

Amyloid-β 42 peptide (AB42) is a 42 amino acid amyloid-β protein fragment of amyloid precursor protein and is the predominant form of amyloid-β found in Alzheimer's disease patients. AB42 and A40 peptides self-assemble into interlaced amyloid fibrils. 50 μM fluorescently-labeled AB42 oligomers and fibrils were added to tissue culture medium containing transduced cell populations by diluting to 1:25 ratio. After 1.5 hrs, transduced cells were washed twice with PBS to remove unassociated fibrillar AB. Phagocytosis was determined as the percentage of fluorescent-positive cells within the cell population.

After incubation, the plate was centrifuged and the media replaced with

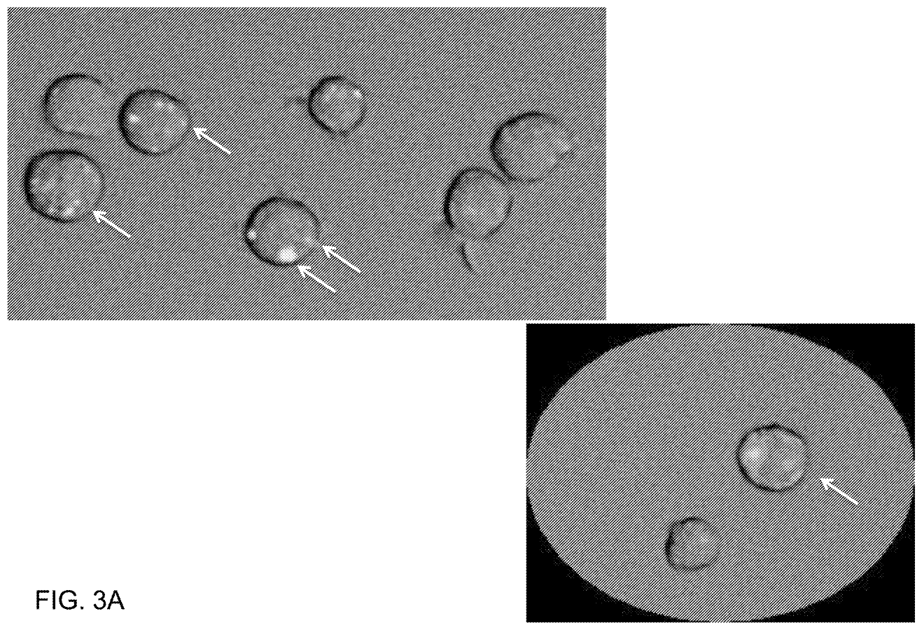

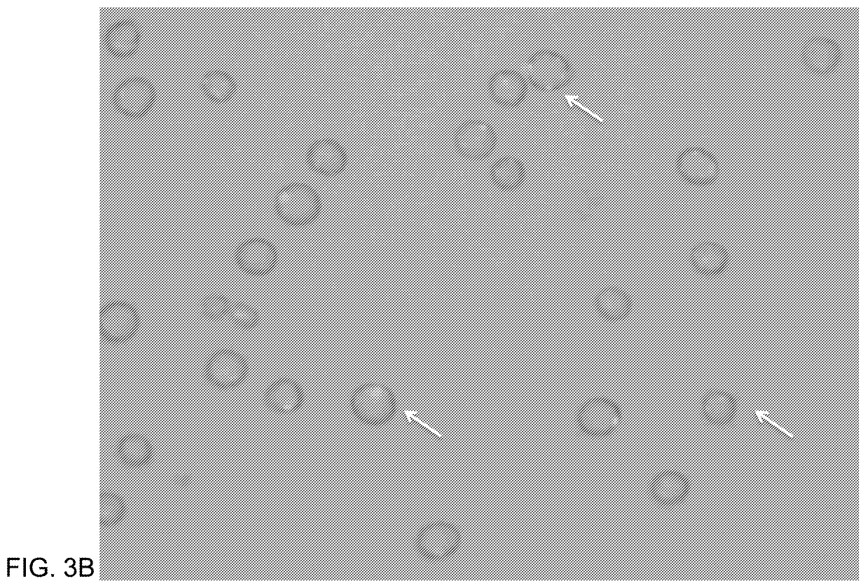

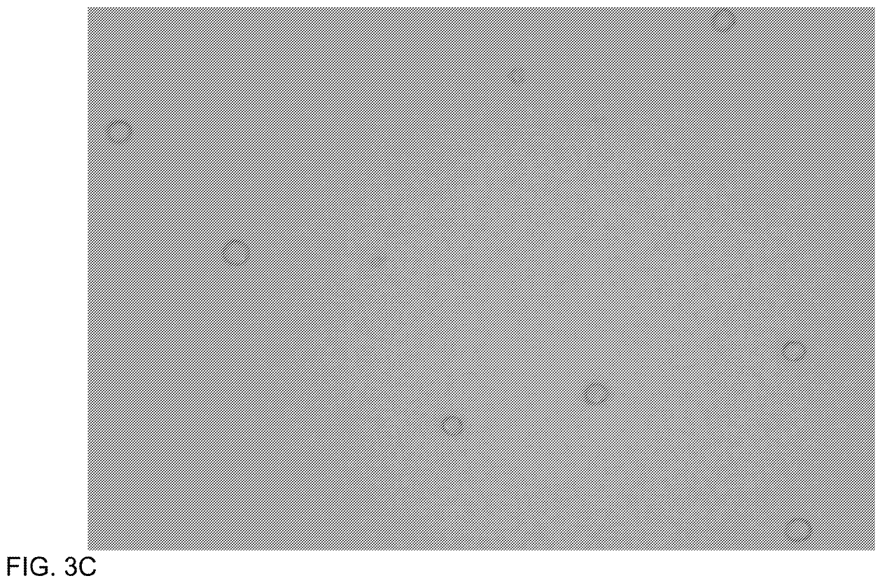

PBS supplemented with 2% fetal bovine serum, pH 9. The 96 well plate was then viewed using KEYENCE BZ-X710 fluorescence microscope, 20× objective. Ba/F3 cells transduced with pLenti vector expressing truncated EGFR were used as a negative control. Fluorescent microscopy showed engulfment of pathogenic AB42 peptide by CER64⁺ Ba/F3 cells at 1.5 hours, 3 hours, and 24 hours post-incubation (see, FIGS. 3A, 3B). tEGFR⁺ Ba/F3 control cells did not exhibit engulfment of AB42 peptide (see, FIG. 3C, 1.5 hours post-incubation).

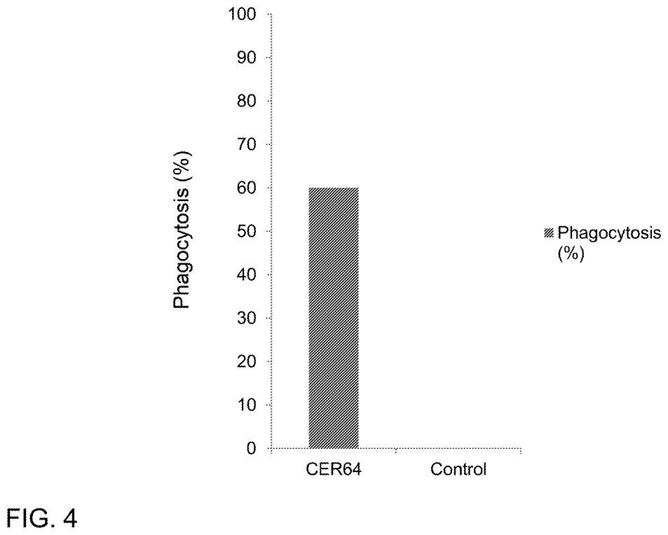

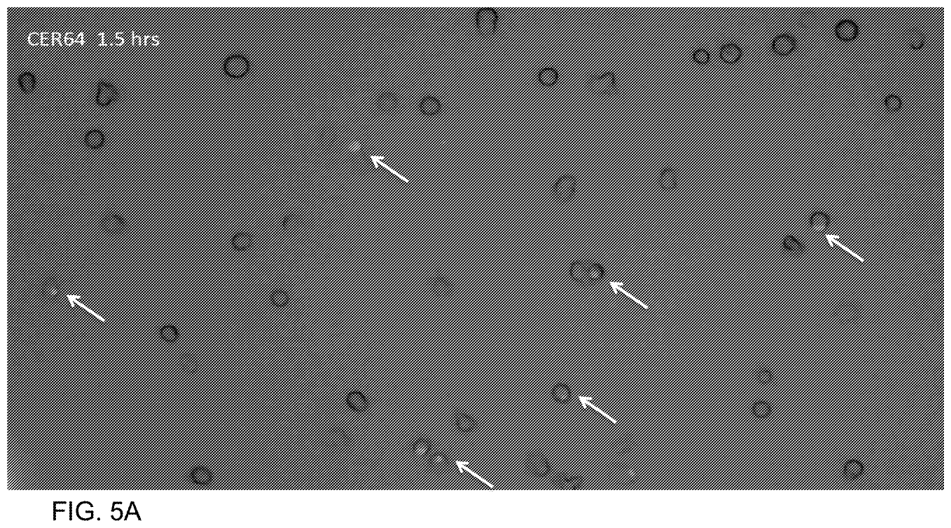

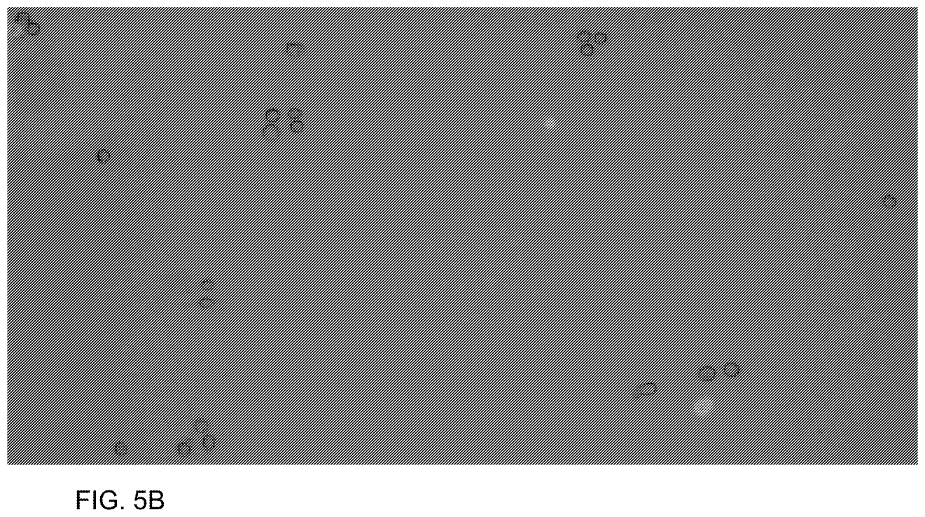

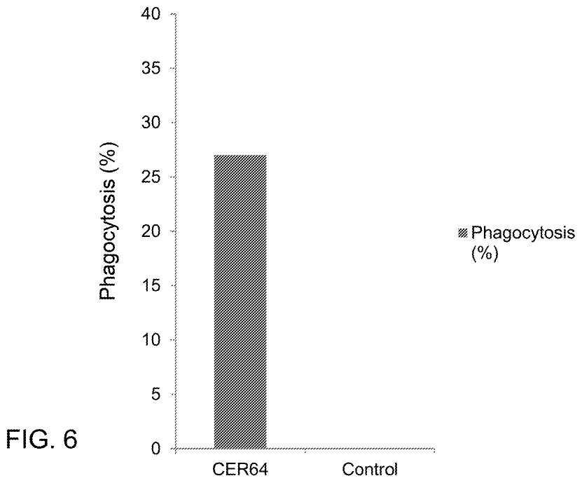

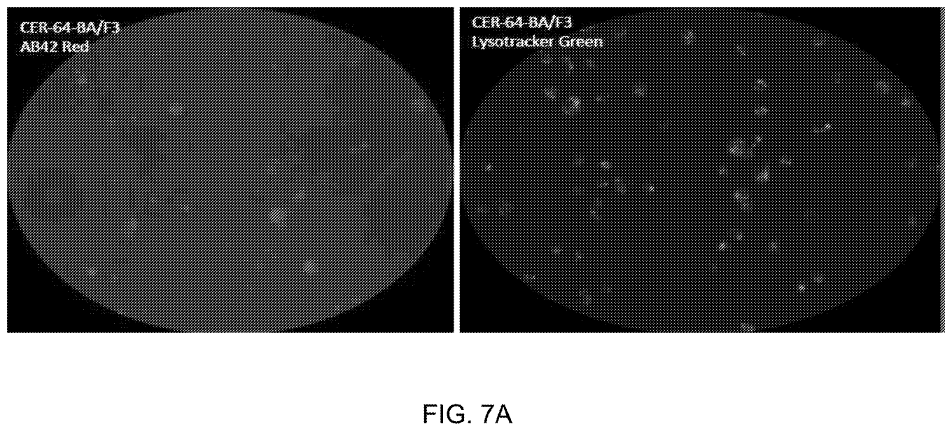

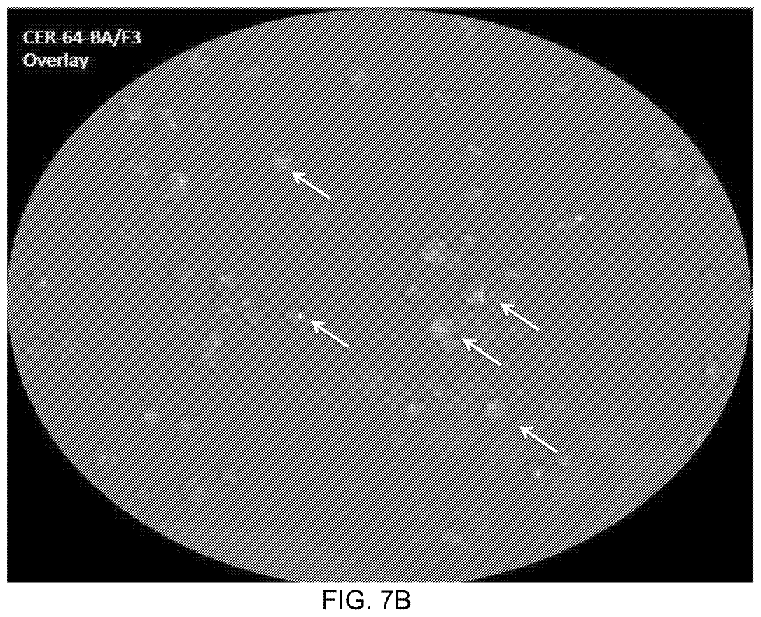

LysoTracker green, which stains acidic compartments (e.g., lysosomes) in live cells green, was added 5 minutes prior to the end of incubation period. Co-localization of internalized pHrodo red labeled AB42 with LysoTracker green vesicles can be visualized by the overlay of these 2 images (see, FIGS. 4A, 4B). Co-localization of red and green fluorescence gives rise to yellow/orange fluorescence in the merged images (see, FIG. 4B), indicating pHrodo-labeled extracellular target AB42 peptides have been internalized into lysosomes, leading to rapid acidification and degradation of the AB42 peptides.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 62/649,472, filed Mar. 28, 2018, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A chimeric engulfment receptor (CER) comprising a single chain chimeric protein, the single chain chimeric protein comprising:
an extracellular domain comprising a binding domain that binds a neurodegenerative disease antigen;
an engulfment signaling domain comprising a homeostatic engulfment signaling domain; and
a transmembrane domain positioned between and connecting the extracellular domain and the engulfment signaling domain.

2. The CER of claim 1, wherein the binding domain comprises a scFv.

3. The CER of claim 1 or 2, wherein the extracellular domain further comprises an extracellular spacer domain positioned between the binding domain and the transmembrane domain.

4. The CER of claim 3, wherein the extracellular spacer domain comprises an immunoglobulin hinge region, an extracellular region of type 1 membrane proteins, a stalk region of a type II C-lectin, an immunoglobulin constant domain, a TLR juxtamembrane domain, or a fragment thereof.

5. The CER of claim any one of claims 1-4, wherein the homeostatic engulfment signaling domain comprises a MERTK, Tyro3, Axl, ELMO, or MRC1 signaling domain.

6. The CER of claim 5, wherein the homeostatic engulfment signaling domain comprises a MERTK signaling domain comprising an amino acid sequence of SEQ ID NO:6, a Tyro3 signaling domain comprising an amino acid sequence of SEQ ID NO:7, an MRC1 signaling domain comprising an amino acid sequence of SEQ ID NO:5, an ELMO signaling domain comprising an amino acid sequence of SEQ ID NO:9, or an Axl signaling domain comprising an amino acid sequence of SEQ ID NO:8.

7. The CER of any one of claims 1-6, wherein the extracellular spacer domain comprises an IgG1, IgG2, IgG3, IgG4, IgA, or IgD hinge region.

8. The CER of claim 7, wherein the extracellular spacer domain comprises a modified IgG4 hinge region comprising an amino acid sequence of SEQ ID NO: 3.

9. The CER of any one of claims 1-8, wherein the transmembrane domain comprises a Tim1, Tim4, Tim3, FcR, CD8a, CD28, MERTK, Axl, Tyro3, CD4, DAP12, MRC1, or TLR transmembrane domain.

10. The CER of any one of claims 1-9, wherein the engulfment signaling domain comprises a primary homeostatic engulfment signaling domain and a secondary engulfment signaling domain.

11. The CER of claim 10, wherein the secondary engulfment signaling domain comprises MERTK, Tyro3, Axl, ELMO, MRC1, Traf6, Syk, MyD88, PI3K, FcϵRIγ, FcγR1, FcγR2A, FcγR2C, FcγR3A, BAFF-R, DAP12, NFAM1, CD79b, TLR1, TLR2, TLR3, TLR4, TLR5, TLR6, TLR7, TLR8, TLR9, Traf2, Traf3 signaling domain.

12. The CER of claim 11, wherein the secondary engulfment signaling domain comprises a MRC1 signaling domain comprising an amino acid sequence of SEQ ID NO:5, a MERTK signaling domain comprising an amino acid sequence of SEQ ID NO:6, a Tyro3 signaling domain comprising an amino acid sequence of SEQ ID NO:7, an Axl signaling domain comprising an amino acid sequence of SEQ ID NO:8, an ELMO signaling domain comprising an amino acid sequence of SEQ ID NO:9, a Traf6 signaling domain comprising an amino acid sequence of SEQ ID NO:10, a Syk signaling domain comprising an amino acid sequence of SEQ ID NO:11, a MyD88 signaling domain comprising an amino acid sequence of SEQ ID NO:12, a FcϵRIγ signaling domain comprising an amino acid sequence of SEQ ID NO:14, a FcγR1 signaling domain comprising an amino acid sequence of SEQ ID NO:15, a FcγR2A signaling domain comprising an amino acid sequence of SEQ ID NO:16, a FcγR2C signaling domain comprising an amino acid sequence of SEQ ID NO:17, a FcγR3A signaling domain comprising an amino acid sequence of SEQ ID NO:18, a BAFF-R signaling domain comprising an amino acid sequence of SEQ ID NO:19, a DAP12 signaling domain comprising an amino acid sequence of SEQ ID NO:20, a NFAM1 signaling domain comprising an amino acid sequence of SEQ ID NO:21, a CD79b signaling domain comprising an amino acid sequence of SEQ ID NO:22, a TLR1 signaling domain comprising an amino acid sequence of SEQ ID NO:23, a TLR2 signaling domain comprising an amino acid sequence of SEQ ID NO:24, a TLR3 signaling domain comprising an amino acid sequence of SEQ ID NO:25, a TLR4 signaling domain comprising an amino acid sequence of SEQ ID NO:26, a TLR5 signaling domain comprising an amino acid sequence of SEQ ID NO:27, a TLR6 signaling domain comprising an amino acid sequence of SEQ ID NO:28, a TLR7 signaling domain comprising an amino acid sequence of SEQ ID NO:29, a TLR8 signaling domain comprising an amino acid sequence of SEQ ID NO:30, a TLR9 signaling domain comprising an amino acid sequence of SEQ ID NO:31, a Traf2 signaling domain comprising an amino acid sequence of SEQ ID NO:32, or a Traf3 signaling domain comprising an amino acid sequence of SEQ ID NO:33.

13. The CER of any one of claims 10-12, wherein the primary homeostatic engulfment signaling domain and secondary engulfment signaling domain are the same or different.

14. The CER of any one of claims 1-13, wherein signaling by the engulfment signaling domain induces expression of an anti-inflammatory cytokine, an immunosuppressive cytokine, or both.

15. The CER of claim 14, wherein the anti-inflammatory or immunosuppressive cytokine is TGF-β, IL-10, or both.

16. The CER of any one of claims 1-15, wherein the neurodegenerative disease antigen is amyloid-β peptide, Tau, beta-secretase, apolipoprotein E4 (ApoE4), alpha-synuclein, leucine rich repeat kinase 2 (LRRK2), presenlin 1, presenilin 2, parkin, gamma secretase, amyloid precursor protein (APP), beta-secretase (BACE1), mutated huntingtin protein (mHTT), Cu,Zn-superoxide dismutase-1 (SOD1), TAR DNA-binding protein 43 (TDP-43), p75 neurotrophin receptor (p75NTR), semaphorin 4D (SEMA4D), ataxin-2, protease-resistant prion protein (PrP^{res}), or pathogenic prion protein (PrP^{Sc}).

17. The CER of any one of claims 1-15, wherein the binding domain comprises a β-amyloid specific scFv comprising an amino acid sequence as set forth in SEQ ID NO:2.

18. The CER of claim 1, comprising:
an extracellular domain comprising: a binding domain comprising a scFv specific to β-amyloid and an extracellular spacer comprising an IgG4 hinge region;
an engulfment signaling domain comprising a MERTK signaling domain;
a transmembrane domain comprising a Tim4 transmembrane domain positioned between and connecting the extracellular domain and the engulfment signaling domain;
wherein the extracellular spacer domain is positioned between the binding domain and the transmembrane domain.

19. The CER of claim 1, comprising:
an extracellular domain comprising: a binding domain comprising a scFv specific to β-amyloid and an extracellular spacer comprising an IgG4 hinge region;
an engulfment signaling domain comprising an Axl signaling domain;
a transmembrane domain comprising a Tim4 transmembrane domain positioned between and connecting the extracellular domain and the engulfment signaling domain;
wherein the extracellular spacer domain is positioned between the binding domain and the transmembrane domain.

20. The CER of claim 18, comprising an amino acid sequence of SEQ ID NO:64.

21. The CER of claim 19, comprising an amino acid sequence of SEQ ID NO:66.

22. A polynucleotide encoding a CER according to any one of claims 1-21.

23. The polynucleotide of claim 22, further comprising a sequence encoding a transduction marker, a suicide gene or both.

24. A vector comprising a polynucleotide according to claim 22 or 23.

25. The vector of claim 24, wherein the vector is a multicistronic vector.

26. The vector of claim 24 or 25, wherein the vector is a viral vector, a modified mRNA vector, or a transposon-mediated gene transfer vector.

27. The vector of claim 26, wherein the viral vector is a retroviral vector or a lentiviral vector.

28. A host cell comprising: a CER according to any one of claims 1-21, a polynucleotide according to claim 22 or 23, or a vector according to any one of claims 24-27.

29. The host cell of claim 28, wherein the host cell is a T cell, a natural killer cell, a B cell, a lymphoid precursor cell, including common lymphocyte precursor cells, an antigen presenting cell, a dendritic cell, a Langerhans cell, a myeloid precursor cell, a mature myeloid cell, a monocyte, a macrophage, a microglial cell, or any combination thereof.

30. The host cell of claim 29, wherein the T cell is a CD4⁺, CD8⁺, naïve (CD45 RA+, CCR7+, CD62L+, CD27+, CD45RO−), central memory (CD45RO⁺, CD62L⁺, CD8⁺), effector memory (CD45RA+, CD45RO−, CCR7−, CD62L−, CD27−), virus-specific, mucosal-associated invariant (MAIT), γδ (gd), natural killer, tissue resident T cell, or any combination thereof.

31. The host cell of claim 29, wherein the B cell is a naïve B cell, plasma cell, regulatory B cell, marginal zone B cell, follicular B cell, lymphoplasmacytoid cell, plasmablast cell, memory B cell, or any combination thereof.

32. The host cell of any one of claims 28-31, wherein the host cell is a human cell.

33. The host cell of any one of claims 28-32, wherein the host cell exhibits engulfment activity when the extracellular domain of the CER binds to the targeted neurodegenerative disease antigen.

34. A population of host cells according to any one of claims 28-33.

35. The population of host cells of claim 34, wherein the population of host cells expresses the same CER.

36. The population of host cells of claim 34, wherein the population of host cells expresses two or more different CERs.

37. A pharmaceutical composition comprising a polynucleotide of claim 22 or 23, a vector according to any one of claims 24-27, a host cell according to any one of claims 28-33, or a population of host cells according to any one of claims 34-36, and a pharmaceutically acceptable excipient.

38. A method of treating a subject having a neurodegenerative disease comprising administering to the subject an effective amount of a host cell according to any one of claims 28-33, a population of host cells according to any one of claims 34-36, or a pharmaceutical composition of claim 37.

39. The method of claim 38, wherein the host cell is an autologous cell.

40. The method of claim 38, wherein the host cell is an allogeneic cell.

41. The method according to any one of claims 38-40, wherein the neurodegenerative disease is Alzheimer's disease (AD), amyotrophic lateral sclerosis (ALS), Huntington's disease, Parkinson's disease, frontotemporal lobar degeneration, or a prion disease.

42. The method of any one of claims 38-41, further comprising administration of a second therapeutic agent.


SEQUENCE LISTING (90 sequences)

SEQ ID NO: 1 - protein, 42 aa, Homo sapiens [sequence omitted]

SEQ ID NO: 2 - protein, 247 aa, Artificial Sequence
  BIIB037 scFv binding domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRGGGGSGGGGSGGGGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 3 - protein, 12 aa, Artificial Sequence
  modified IgG4 hinge
ESKYGPPCPPCP

SEQ ID NO: 4 - protein, 13 aa, Homo sapiens
  TLR4 juxtamembrane domain
PVLSLNITCQMNK

SEQ ID NO: 5 - protein, 46 aa, Homo sapiens
  MRC1 signaling domain
YKKRRVHLPQEGAFENTLYFNSQSSPGTSDMKDLVGNIEQNEHSVI

SEQ ID NO: 6 - protein, 473 aa, Homo sapiens
  MERTK signaling domain
KRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 7 - protein, 440 aa, Homo sapiens
  Tyro3 signaling domain
LRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 8 - protein, 422 aa, Homo sapiens
  Axl signaling domain
HRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 9 - protein, 727 aa, Homo sapiens
  ELMO signaling domain
MPPPADIVKVAIEWPGAYPKLMEIDQKKPLSAIIKEVCDGWSLANHEYFALQHADSSNFYITEKNRNEIKNGTILRLTTSPAQNAQQLHERIQSSSMDAKLEALKDLASLSRDVTFAQEFINLDGISLLTQMVESGTERYQKLQKIMKPCFGDMLSFTLTAFVELMDHGIVSWDTFSVAFIKKIASFVNKSAIDISILQRSLAILESMVLNSHDLYQKVAQEITIGQLIPHLQGSDQEIQTYTIAVINALFLKAPDERRQEMANILAQKQLRSIILTHVIRAQRAINNEMAHQLYVLQVLTFNLLEDRMMTKMDPQDQAQRDIIFELRRIAFDAESEPNNSSGSMEKRKSMYTRDYKKLGFINHVNPAMDFTQTPPGMLALDNMLYFAKHHQDAYIRIVLENSSREDKHECPFGRSSIELTKMLCEILKVGELPSETCNDFHPMFFTHDRSFEEFFCICIQLLNKTWKEMRATSEDFNKVMQVVKEQVMRALTTKPSSLDQFKSKLQNLSYTEILKIRQSERMNQEDFQSRPILELKEKIQPEILELIKQQRLNRLVEGTCFRKLNARRRQDKFWYCRLSPNHKVLHYGDLEESPQGEVPHDSLQDKLPVADIKAVVTGKDCPHMKEKGALKQNKEVLELAFSILYDSNCQLNFIAPDKHEYCIWTDGLNALLGKDMMSDLTRNDLDTLLSMEIKLRLLDLENIQIPDAPPPIPKEPSNYDFVYDCN

SEQ ID NO: 10 - protein, 522 aa, Homo sapiens
  Traf6 signaling domain - full length
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAQAVHSLSVIPDSGYISEVRNFQETIHQLEGRLVRQDHQIRELTAKMETQSMYVSELKRTIRTLEDKVAEIEAQQCNGIYIWKIGNFGMHLKCQEEEKPVVIHSPGFYTGKPGYKLCMRLHLQLPTAQRCANYISLFVHTMQGEYDSHLPWPFQGTIRLTILDQSEAPVRQNHEEIMDAKPELLAFQRPTIPRNPKGFGYVTFMHLEALRQRTFIKDDTLLVRCEVSTRFDMGSLRREGFQPRSTDAGV

SEQ ID NO: 11 - protein, 261 aa, Homo sapiens
  Syk signaling domain
TLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYY

SEQ ID NO: 12 - protein, 296 aa, Homo sapiens
  MyD88 signaling domain
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 13 - skipped

SEQ ID NO: 14 - protein, 42 aa, Homo sapiens
  FcepsilonR1gamma signaling domain
RLKIQVRKAAITSYEKSDGVYTGLSTRNQETYETLKHEKPPQ

SEQ ID NO: 15 - protein, 61 aa, Homo sapiens
  FcgammaR1 signaling domain
RKELKRKKKWDLEISLDSGHEKKVISSLQEDRHLEEELKCQEQKEEQLQEGVHRKEPQGAT

SEQ ID NO: 16 - protein, 77 aa, Homo sapiens
  FcgammaR2A signaling domain
CRKKRISANSTDPVKAAQFEPPGRQMIAIRKRQLEETNNDYETADGGYMTLNPRAPTDDDKNIYLTLPPNDHVNSNN

SEQ ID NO: 17 - protein, 77 aa, Homo sapiens
  FcgammaR2c signaling domain
CRKKRISANSTDPVKAAQFEPPGRQMIAIRKRQPEETNNDYETADGGYMTLNPRAPTDDDKNIYLTLPPNDHVNSNN

SEQ ID NO: 18 - protein, 25 aa, Homo sapiens
  FcgammaR3A signaling domain
KTNIRSSTRDWKDHKFKWRKDPQDK

SEQ ID NO: 19 - protein, 85 aa, Homo sapiens
  BAFF-R signaling domain
SWRRRQRRLRGASSAEAPDGDKDAPEPLDKVIILSPGISDATAPAWPPPGEDPGTTPPGHSVPVPATELGSTELVTTKTAGPEQQ

SEQ ID NO: 20 - protein, 52 aa, Homo sapiens
  DAP12 signaling domain
YFLGRLVPRGRGAAEAATRKQRITETESPYQELQGQRSDVYSDLNTQRPYYK

SEQ ID NO: 21 - protein, 86 aa, Homo sapiens
  NFAM1 signaling domain
LWNKKRMRGPGKDPTRKCPDPRSASSPKQHPSESVYTALQRRETEVYACIENEDGSSPTAKQSPLSQERPHRFEDDGELNLVYENL

SEQ ID NO: 22 - protein, 29 aa, Homo sapiens
  CD79b signaling domain 185-213
DSKAGMEEDHTYEGLDIDQTATYEDIVTL

SEQ ID NO: 23 - protein, 185 aa, Homo sapiens
  TLR1 signaling domain
SYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 24 - protein, 175 aa, Homo sapiens
  TLR2 signaling domain
HRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 25 - protein, 179 aa, Homo sapiens
  TLR3 signaling domain
EGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 26 - protein, 187 aa, Homo sapiens
  TLR4 signaling domain
KFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 27 - protein, 198 aa, Homo sapiens
  TLR5 signaling domain
TKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 28 - protein, 189 aa, Homo sapiens
  TLR6 signaling domain
YLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 29 - protein, 189 aa, Homo sapiens
  TLR7 signaling domain
HLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 30 - protein, 193 aa, Homo sapiens
  TLR8 signaling domain
HHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 31 - protein, 193 aa, Homo sapiens
  TLR9 signaling domain
GWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 32 - protein, 303 aa, Homo sapiens
  TRAF2 signaling domain
MAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 33 - protein, 274 aa, Homo sapiens
  TRAF3 signaling domain
MESSKKMDSPGALQTNPPLKLHTDRSAGTPVFVPEQGGYKEKFVKTVEDKYKCEKCHLVLCSPKQTECGHRFCESCMAALLSSSSPKCTACQESIVKDKVFKDNCCKREILALQIYCRNESRGCAEQLMLGHLLVHLKNDCHFEELPCVRPDCKEKVLRKDLRDHVEKACKYREATCSHCKSQVPMIALQKHEDTDCPCVVVSCPHKCSVQTLLRSELSAHLSECVNAPSTCSFKRYGCVFQGTNQQIKAHEASSAVQHVNLLKEWSNSLEKKV

SEQ ID NO: 34 - protein, 155 aa, Homo sapiens
  truncated MyD88 signaling domain without TIR domain
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 35 - protein, 274 aa, Homo sapiens
  truncated TRAF6 signaling domain
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 36 - protein, 29 aa, Homo sapiens
  truncated NFAM1 signaling domain
SSPKQHPSESVYTALQRRETEVYACIENE

SEQ ID NO: 37 - protein, 21 aa, Homo sapiens
  Tim1 transmembrane domain
IYAGVCISVLVLLALLGVIIA

SEQ ID NO: 38 - protein, 21 aa, Homo sapiens
  Tim4 transmembrane domain
LLMIIAPSLGFVLFALFVAFL

SEQ ID NO: 39 - protein, 21 aa, Homo sapiens
  Tim3 transmembrane domain
IYIGAGICAGLALALIFGALI

SEQ ID NO: 40 - protein, 21 aa, Homo sapiens
  FcgammaR1 transmembrane domain
VLFYLAVGIMFLVNTVLWVTI

SEQ ID NO: 41 - protein, 23 aa, Homo sapiens
  FcgammaR2A transmembrane domain
IIVAVVIATAVAAIVAAVVALIY

SEQ ID NO: 42 - protein, 23 aa, Homo sapiens
  FcgammaR2B2 transmembrane domain
SSSPMGIIVAVVTGIAVAAIVAA

SEQ ID NO: 43 - protein, 23 aa, Homo sapiens
  FcgammaR2C transmembrane domain
IIVAVVTGIAVAAIVAAVVALIY

SEQ ID NO: 44 - protein, 21 aa, Homo sapiens
  FcgammaR3A transmembrane domain
VSFCLVMVLLFAVDTGLYFSV

SEQ ID NO: 45 - protein, 21 aa, Homo sapiens
  FcepsilonR1gamma transmembrane domain
LCYILDAILFLYGIVLTLLYC

SEQ ID NO: 46 - protein, 19 aa, Homo sapiens
  FcalphaR1 transmembrane domain
LIRMAVAGLVLVALLAILV

SEQ ID NO: 47 - protein, 21 aa, Homo sapiens
  CD8a transmembrane domain
IYIWAPLAGTCGVLLLSLVIT

SEQ ID NO: 48 - protein, 27 aa, Homo sapiens
  CD28 transmembrane domain
FWVLVVVGGVLACYSLLVTVAFIIFWV

SEQ ID NO: 49 - protein, 21 aa, Homo sapiens
  MERTK transmembrane domain
FGCFCGFILIGLILYISLAIR

SEQ ID NO: 50 - protein, 21 aa, Homo sapiens
  Axl transmembrane domain
YVLLGAVVAAACVLILALFLV

SEQ ID NO: 51 - protein, 21 aa, Homo sapiens
  Tyro3 transmembrane domain
VPVVLGVLTALVTAAALALIL

SEQ ID NO: 52 - protein, 22 aa, Homo sapiens
  CD4 transmembrane domain
MALIVLGGVAGLLLFIGLGIFF

SEQ ID NO: 53 - protein, 21 aa, Homo sapiens
  DAP12 transmembrane domain
GVLAGIVMGDLVLTVLIALAV

SEQ ID NO: 54 - protein, 21 aa, Homo sapiens
  MRC1 transmembrane domain
GVVIIVILLILTGAGLAAYFF

SEQ ID NO: 55 - protein, 21 aa, Homo sapiens
  TLR1 transmembrane domain
LLIVTIVATMLVLAVTVTSLC

SEQ ID NO: 56 - protein, 21 aa, Homo sapiens
  TLR2 transmembrane domain
ALVSGMCCALFLLILLTGVLC

SEQ ID NO: 57 - protein, 21 aa, Homo sapiens
  TLR3 Transmembrane domain
FFMINTSILLIFIFIVLLIHF

SEQ ID NO: 58 - protein, 21 aa, Homo sapiens
  TLR4 transmembrane domain
TIIGVSVLSVLVVSVVAVLVY

SEQ ID NO: 59 - protein, 21 aa, Homo sapiens
  TLR5 transmembrane domain
FSLFIVCTVTLTLFLMTILTV

SEQ ID NO: 60 - protein, 21 aa, Homo sapiens
  TLR6 transmembrane domain
ALVSGMCCALFLLILLTGVLC

SEQ ID NO: 61 - protein, 21 aa, Homo sapiens
  TLR7 transmembrane domain
LILFSLSISVSLFLMVMMTAS

SEQ ID NO: 62 - protein, 21 aa, Homo sapiens
  TLR8 transmembrane domain
AVILFFFTFFITTMVMLAALA

SEQ ID NO: 63 - protein, 21 aa, Homo sapiens
  TLR9 transmembrane domain
FALSLLAVALGLGVPMLHHLC

SEQ ID NO: 64 - skipped

SEQ ID NO: 65 - protein, 756 aa, Artificial Sequence
  CER64 (BIIB037 scFv-IgG4 hinge-Tim4 transmembrane-MERTK signaling domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRGGGGSGGGGSGGGGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSESKYGPPCPPCPILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLM

SEQ ID NO: 66 - protein, 702 aa, Artificial Sequence
  CER65 (BIIB037 scFv-IgG4 hinge-Tim4 transmembrane domain - Axl signaling domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRGGGGSGGGGSGGGGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSESKYGPPCPPCPILIIACCVGFVLMVLLFLAFLHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 67 - skipped

SEQ ID NO: 68 - protein, 22 aa, Homo sapiens
  GM-CSF signal peptide sequence
MLLLVTSLLLCELPHPAFLLIP

SEQ ID NO: 69 - protein, 24 aa, Homo sapiens
  Tim4 signal peptide sequence
MSKEPLILWLMIEFWWLYLTPVTS

SEQ ID NO: 70 - protein, 18 aa, Artificial Sequence
  T2A self-cleaving peptide
EGRGSLLTCGDVEENPGP

SEQ ID NO: 71 - protein, 19 aa, Artificial Sequence
  P2A self-cleaving peptide
ATNFSLLKQAGDVEENPGP

SEQ ID NO: 72 - protein, 20 aa, Artificial Sequence
  E2A self-cleaving peptide
QCTNYALLKLAGDVESNPGP

SEQ ID NO: 73 - protein, 22 aa, Artificial Sequence
  F2A self-cleaving peptide
VKQTLNFDLLKLAGDVESNPGP

SEQ ID NO: 74 - protein, 285 aa, Homo sapiens
  truncated EGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCH

SEQ ID NO: 75 - protein, 192 aa, Homo sapiens
  Rac1
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 76 - protein, 215 aa, Homo sapiens
  Rab5
MASRGATRPNGPNTGNKICQFKLVLLGESAVGKSSLVLRFVKGQFHEFQESTIGAAFLTQTVCLDDTTVKFEIWDTAGQERYHSLAPMYYRGAQAAIVVYDITNEESFARAKNWVKELQRQASPNIVIALSGNKADLANKRAVDFQEAQSYADDNSLLFMETSAKTSMNVNEIFMAIAKKLPKNEPQNPGANSARGRGVDLTEPTQPTRNQCCSN

SEQ ID NO: 77 - protein, 207 aa, Homo sapiens
  Rab7
MTSRKKVLLKVIILGDSGVGKTSLMNQYVNKKFSNQYKATIGADFLTKEVMVDDRLVTMQIWDTAGQERFQSLGVAFYRGADCCVLVFDVTAPNTFKTLDSWRDEFLIQASPRDPENFPFVVLGNKIDLENRQVATKRAQAWCYSKNNIPYFETSAKEAINVEQAFQTIARNALKQETEVELYNEFPEPIKLDKNDRAKASAESCSC

SEQ ID NO: 78 - protein, 184 aa, Homo sapiens
  Rap1
MREYKLVVLGSGGVGKSALTVQFVQGIFVEKYDPTIEDSYRKQVEVDCQQCMLEILDTAGTEQFTAMRDLYMKNGQGFALVYSITAQSTFNDLQDLREQILRVKDTEDVPMILVGNKCDLEDERVVGKEQGQNLARQWCNCAFLESSAKSKINVNEIFYDLVRQINRKTPVEKKKPKKKSCLLL

SEQ ID NO: 79 - protein, 193 aa, Homo sapiens
  RhoA
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 80 - protein, 191 aa, Homo sapiens
  CDC42
MQTIKCVVVGDGAVGKTCLLISYTTNKFPSEYVPTVFDNYAVTVMIGGEPYTLGLFDTAGQEDYDRLRPLSYPQTDVFLVCFSVVSPSSFENVKEKWVPEITHHCPKTPFLLVGTQIDLRDDPSTIEKLAKNKQKPITPETAEKLARDLKAVKYVECSALTQKGLKNVFDEAILAALEPPEPKKSRRCVLL

SEQ ID NO: 81 - protein, 21 aa, Mus musculus
  Tim4 transmembrane domain
ILIIACCVGFVLMVLLFLAFL

SEQ ID NO: 82 - protein, 279 aa, Mus musculus
  Tim4 binding domain, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQT

SEQ ID NO: 83 - protein, 22 aa, Mus musculus
  Tim4 signal peptide
MSKGLLLLWLVTELWWLYLTPA

SEQ ID NO: 84 - protein, 45 aa, Homo sapiens
  CD79b signaling domain 185-229
DSKAGMEEDHTYEGLDIDQTATYEDIVTLRTGEVKWSVGEHPGQE

SEQ ID NO: 85 - protein, 476 aa, Mus musculus
  MERTK signaling domain
ALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLM

SEQ ID NO: 86 - protein, 141 aa, Homo sapiens
  MyD88 TIR domain
HMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 87 - protein, 24 aa, Artificial Sequence
  T2A self-cleaving peptide variant
LEGGGEGRGSLLTCGDVEENPGPR

SEQ ID NO: 88 - protein, 23 aa, Artificial Sequence
  T2A self-cleaving peptide variant
LEGGGEGRGSLLTCGDVEENPGP

SEQ ID NO: 89 - protein, 19 aa, Artificial Sequence
  T2A self-cleaving peptide variant
EGRGSLLTCGDVEENPGPR

SEQ ID NO: 90 - protein, 27 aa, Artificial Sequence
  P2A self-cleaving peptide variant
RAKRSGSGATNFSLLKQAGDVEENPGP